(19) United States
(12) Patent Application Publication
(10) Pub. No.: US 2026/0263798 A1
(43) Pub. Date: Sep. 10, 2026

(54) ELECTROCOCHLEOGRAPHY LOCALIZATION OF CHARACTERISTIC FREQUENCY FOR COCHLEAR IMPLANT MAPPING

(71) Applicants: Ohio State Innovation Foundation, Columbus, OH (US); The University of North Carolina at Chapel Hill, Chapel Hill, NC (US); Washington University, St. Louis, MO (US)

(72) Inventors: Oliver F. ADUNKA, Columbus, OH (US); Craig A. BUCHMAN, Clayton, MO (US); Douglas C. FITZPATRICK, Chapel Hill, NC (US); Amit WALIA, St. Louis, MO (US)

(21) Appl. No.: 18/876,340

(22) PCT Filed: Jun. 22, 2023
(86) PCT No.: PCT/US2023/026001

§ 371 Date: Dec. 18, 2024

Related U.S. Application Data
(60) Provisional application No. 63/354,371, filed on Jun. 22, 2022.

(51) Int. Cl. A61N 1/36; A61N 1/05

(52) U.S. Cl. CPC A61N 1/36039; A61N 1/0541

(57) ABSTRACT

Devices and methods for delivering electrical stimulation using an auditory prosthetic device are described herein. An example method includes delivering an acoustic stimulation to a subject, and recording, using an electrode array inserted into at least a portion of the subject's cochlea, an acoustically-evoked response to the acoustic stimulation. The method also includes analyzing the acoustically-evoked response to determine a response map, where the response map associates respective frequencies of the acoustic stimulation to respective elements (e.g., electrode contacts or channels) of the electrode array and underlying cochlear place. The method further includes delivering, using the electrode array, an electrical stimulation to the subject. The electrical stimulation is delivered via the respective elements of the electrode array based on the previously obtained acoustically evoked response map.

### CROSS-REFERENCE TO RELATED APPLICATIONS

This application claims the benefit of U.S. provisional patent application No. 63/354,371, filed on Jun. 22, 2022, and titled “ELECTROCOCHLEOGRAPHY LOCALIZATION OF CHARACTERISTIC FREQUENCY FOR COCHLEAR IMPLANT MAPPING,” the disclosure of which is expressly incorporated herein by reference in its entirety.

### BACKGROUND

Cochlear implants are the standard of care for hearing restoration in patients with moderate to profound hearing loss that continue to struggle despite using hearing aids. A cochlear implant is an implantable neural stimulator that delivers electrical pulses to the cochlea, to activate the cochlear nerve, in response to sound. For patients with limited residual hearing, electrical stimulation using a cochlear implant can restore sound perception and improve speech understanding in both quiet and noise, as well as music perception and quality of life. While most people that receive a cochlear implant lose some or all their native hearing following surgery, it has also been shown that individuals with substantial residual hearing following cochlear implantation can combine the electrical stimulation of a cochlear implant with acoustic stimulation in the same ear to improve even more, speech recognition abilities in both quiet and noise as well as music appreciation.

### SUMMARY

An example method for delivering electrical stimulation using an auditory prosthetic device is described herein. The method includes delivering an acoustic stimulation to a subject, and recording, using an electrode array inserted into at least a portion of the subject's cochlea, an acoustically-evoked response to the acoustic stimulation. The method also includes analyzing the acoustically-evoked responses to determine a response map, where the response map associates respective frequencies and intensities of the acoustic stimulation to respective elements of the electrode array, and thus, the underlying cochlear place. The method further includes delivering, using the electrode array, an electrical stimulation to the subject. The electrical stimulation is delivered via the respective elements of the electrode array based on the response map.

Additionally, the method further includes receiving an acoustic signal recorded by a microphone, converting the acoustic signal to a digital signal, and generating the electrical stimulation based on the digital signal.

Alternatively, or additionally, the electrical stimulation is delivered in a frequency-specific manner to locations of the subject's cochlea via the respective elements of the electrode array.

Alternatively, or additionally, the step of analyzing the acoustically-evoked response to determine the response map includes localizing a plurality of characteristic (or best) frequencies.

Alternatively, or additionally, the step of analyzing the acoustically-evoked response to determine the frequency-specific response map includes analyzing at least one characteristic of the acoustically-evoked response. Optionally, the step of analyzing the acoustically-evoked response to determine the response map further includes analyzing a physiological feature of the subject.

Alternatively, or additionally, the acoustically-evoked response is an electrocochleography (ECochG) signal.

Alternatively, or additionally, the acoustically-evoked response is an early auditory potential.

Alternatively, or additionally, the electrical stimulation is characterized by at least one of a frequency, an amplitude, or a duration.

An example method for localizing characteristic (or best) frequency for cochlear implant mapping is also described herein. The method includes delivering an acoustic stimulation to a subject, and recording, using an electrode array, an acoustically-evoked response to the acoustic stimulation, where the acoustically-evoked response is an electrocochleography (ECochG) signal. The method also includes analyzing the ECochG signal to determine a response map, where the response map associates respective frequencies of the acoustic stimulation to respective elements of the electrode array, and thus the cochlear place (or location).

An example auditory prosthetic device is also described herein. The device includes an electrode array that is configured for insertion into at least a portion of a subject's cochlea, and a processor coupled to the electrode array. The processor is configured to store an acoustically-evoked response map, where the acoustically-evoked response map associates respective frequencies of an acoustic stimulation to respective elements of the electrode array, receives an acoustic signal recorded by a microphone, and convert the acoustic signal to a digital signal. The processor is further configured to generate an electrical stimulation based on the digital signal, and transmit the electrical stimulation to the electrode array. The electrical stimulation is transmitted to the respective elements of the electrode array based on the response map.

Optionally, the auditory prosthetic device is an implantable or semi-implantable device. For example, the auditory prosthetic device can be a cochlear implant.

In some implementations, the techniques described herein relate to a method including: delivering an acoustic stimulation to a subject; obtaining an acoustically-evoked response to the acoustic stimulation; determining one or more stimulus parameters from the acoustically-evoked response; and determining an auditory stimulation protocol for the subject based at least in part on the one or more stimulus parameters.

In some implementations, the techniques described herein relate to a method, wherein the auditory stimulation protocol is used to program an auditory prosthetic device for the subject.

In some implementations, the techniques described herein relate to a method, wherein the stimulus parameters include at least one of an intensity, a frequency, and a cochlea location.

In some implementations, the techniques described herein relate to a method, wherein the auditory prosthetic device includes a plurality of electrodes, and wherein the auditory stimulation protocol includes an assignment of a respective frequency band to one or more electrodes.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates pre-and post-operative audiograms of a patient that has received a cochlear implant in an example described herein. FIG. 1B illustrates the raw ECochG waves measured by the individual cochlear implant electrodes (basal→apical) inside the patient's cochlea in response to the various frequency stimuli (250 Hz-2 KHz). FIG. 1C illustrates the result of a fast Fourier transformation (FFT) algorithm to identify the quantitative response of the waveforms at the various recording electrodes.

FIG. 2A is a flowchart illustrating example operations for delivering electrical stimulation using an auditory prosthetic device according to an implementation described herein.

FIG. 2B is a flowchart illustrating example operations for determining an auditory stimulation protocol for a subject according to an implementation described herein.

FIG. 3A is a block diagram illustrating an auditory prosthetic device according to an implementation described herein.

FIG. 3B is an example system according to an implementation described herein.

FIG. 3C a schematic diagram depicting results from a study that was conducted using the system depicted in FIG. 3B.

FIG. 4 is an example computing device.

FIG. 5A is a graph showing a difference curve that was calculated by subtracting rarefaction from condensation phase stimuli.

FIG. 5B is a schematic diagram depicting Computed tomography (CT) imaging and 3D reconstructions.

FIG. 5C is a graph showing results for developing a frequency-position function for an individual subject's cochlea using the electrophysiologic responses and CT imaging for the location of each electrode.

FIG. 5D is a graph showing results of electrophysiologic measurements repeated in 49 additional subjects.

FIG. 5E, FIG. 5F, FIG. 5G, FIG. 5H, FIGURE I, and FIGURE J are graphs showing results of the impact of intensity stimulus on the frequency-position map.

FIG. 5K is a table showing a comparative analysis of in vivo and Greenwood frequency-position functions.

FIG. 5L shows demographic, audiologic, and imaging information of fifty subjects tested to construct a electrophysiologically-derived frequency-position map.

FIG. 5M, FIG. 5N, and FIG. 50 illustrate stimulus intensity and frequency-position maps generated from a study that was conducted.

FIG. 5P is a schematic diagram depicting pitch-discrimination testing to determine the impact of the presence of electrode on the frequency-position map.

FIG. 5Q, FIG. 5R, FIG. 55, and FIG. 5T are graphs showing results of the pitch-discrimination testing.

FIG. 6A, FIG. 6B, FIG. 6C, FIG. 6D, FIG. 6E, FIG. 6F, and FIG. 6G are graphs depicting the effects of third-window fenestration on the frequency-position map.

FIG. 7A and FIG. 7B are graphs depicting speech-perception performance results following cochlear implantation in relation to frequency-to-place mismatch between in vivo and Greenwood maps, respectively.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for cochlear implants, it will become evident to those skilled in the art that the implementations are not limited thereto, but are applicable for other implantable or semi-implantable devices.

As used herein, the terms “about” or “approximately” when referring to a measurable value such as an amount, a percentage, and the like, is meant to encompass variations of ±20%, ±10%, ±5%, or ±1% from the measurable value.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

Cochlear implants leverage the tonotopic organization of the cochlea to provide electrical stimulation to local nerve fibers (i.e., spiral ganglion cells and dendrites) in a frequency-specific place. Thus, the speech processor of a cochlear implant decodes the acoustic signal into its component frequency and intensity elements and delivers the high frequencies to the basal aspects of the cochlea and the low frequencies to the more apical aspects of the cochlea. This tonotopic arrangement has been known since the early 1900's and forms the basis for using multiple electrodes to stimulate the inner ear with cochlear implants. The relationship of cochlear place to frequency has been defined in both animal and human studies and has been quantified by Greenwood's map. This map provides for a relationship between cochlear location (i.e., place within the cochlea) and frequency place (i.e., the location of maximal basilar membrane movement inside the cochlea-Characteristic frequency (CF) when determined at threshold or Best frequency (BF) when determined at higher stimulation levels).

With recent advances in electrophysiological technology, it has become possible to make measurements inside the cochlea in humans in response to acoustic stimuli. This is termed electrocochleography (ECochG). These ECochG recordings, when carried out using the electrodes of a cochlear implant, can be used to directly measure and thus localize CF or BF as described below. Knowing CF or BF for an individual ear is important as it can help in the mapping of the cochlear implant stimulus location or place in a more specific way. In particular, ECochG can be used to localize CF or BF and then use this CF or BF to map individual frequencies to respective places inside the cochlea.

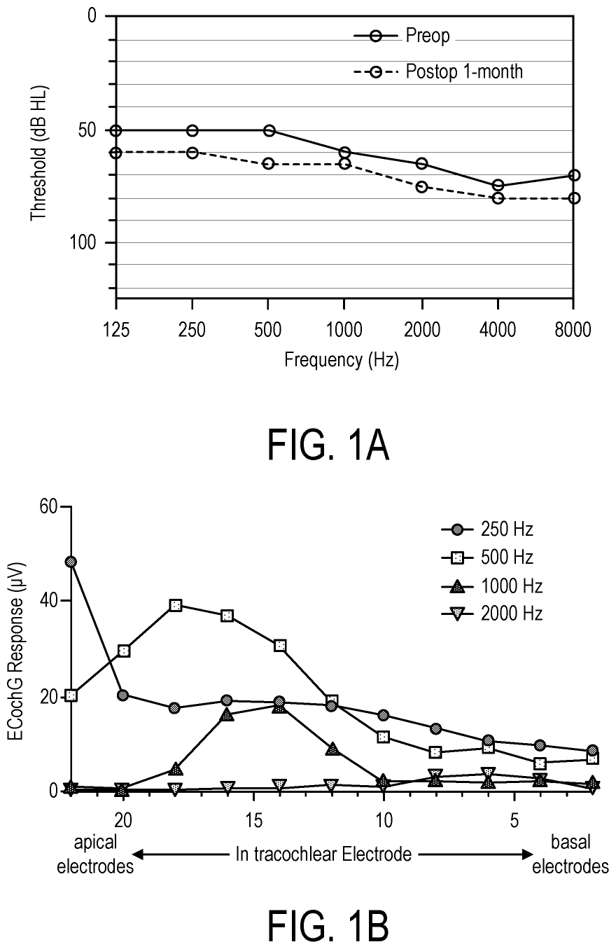

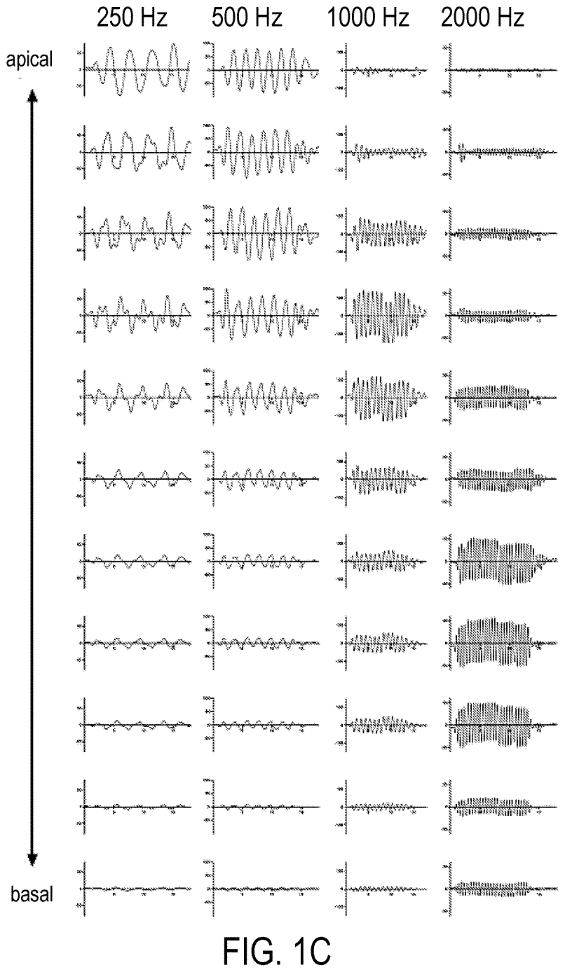

For example, FIG. 1A shows the pre-and post-operative audiogram of a patient that has received a cochlear implant. The individual's hearing is partially preserved after surgery. FIG. 1B shows the raw ECochG waves measured by the individual cochlear implant electrodes (basal→apical) inside the patient's cochlea in response to the various frequency stimuli (250 Hz-2 KHz), FIG. 1C shows the result of a fast Fourier transformation (FFT) algorithm to identify the quantitative response of the waveforms at the various recording electrodes. In this case, BF (i.e., the maximal FFT signal) for 250 Hz is localized at e22 (or deeper) while BF for 500 Hz is at e18, 1000 Hz BF is at e14, and BF for 2000 Hz is at e6. It should be understood that embodiments of the present disclosure are not limited to the FFT-based analysis and other techniques may be employed such as Discrete Cosine Transform (DCT) or Discrete Fourier Transform (DFT).

The devices and methods described herein use BF localization information to assign the cochlear implant electrodes to the various frequencies for electrical stimulation. As an example, a 500 Hz frequency detected by the cochlear implant speech processor in the environment is then converted to electrical pulses of a certain rate, amplitude and duration and delivered to e18 consistent with the ECochG place map defined above. Similarly, 250 Hz, 1000 Hz, and 2000 Hz signals detected by the speech processor microphone are decoded and electrical pulses delivered to e22, e14, and e6, respectively.

Typically, in cochlear implants today, the frequency ranges assigned are preset by the manufacturer, and do not take into account the actual position of each contact after surgery (as shown above). Thus, many patients have a ‘frequency-to-place’ mismatch, resulting in an unnatural sound percept where speech patterns need to be relearned, requiring a substantial listening effort. The devices and methods described herein provide a means to record and interpret physiological responses that remain in the cochlea of each subject to specify a pattern of stimulation to best fit each patient and lead to better and faster success with the implanted device.

### Example Methods

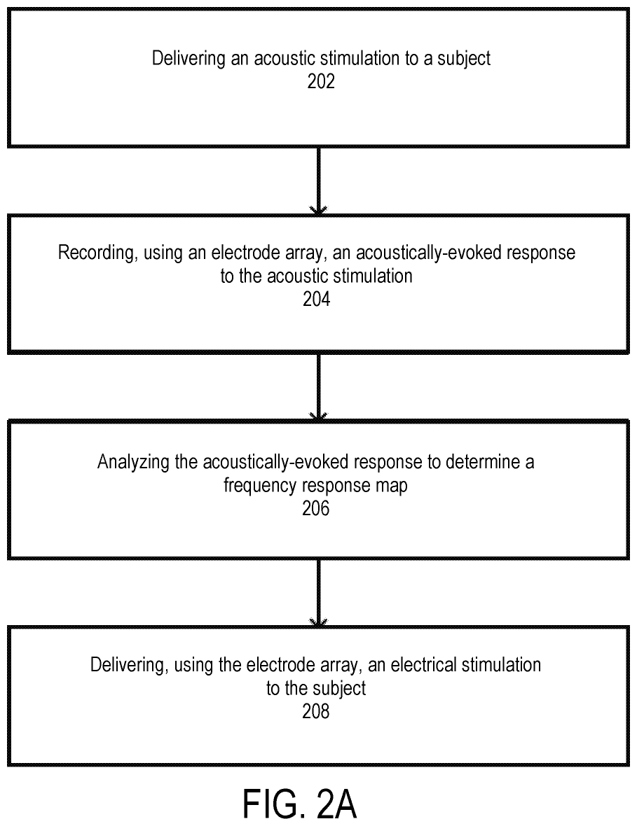

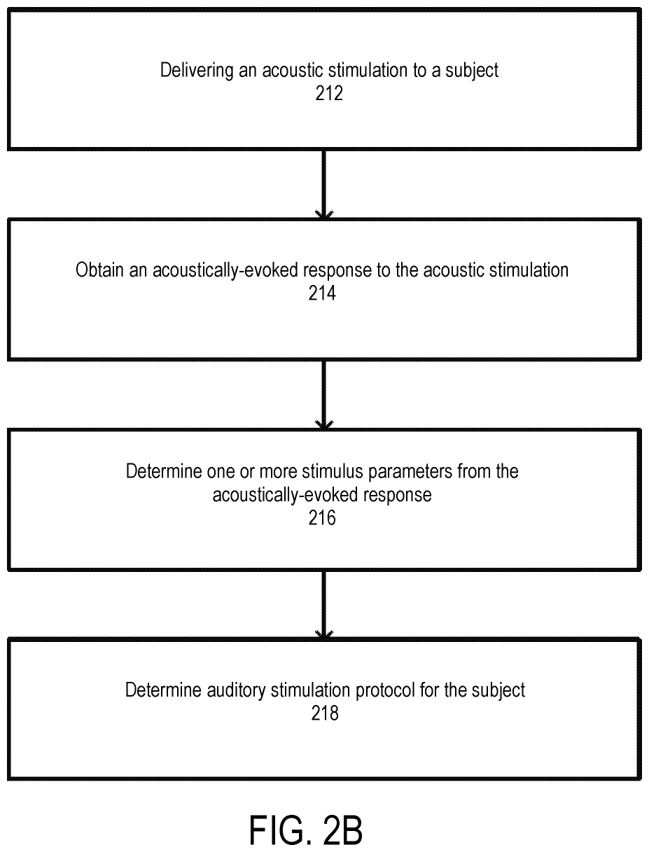

Referring now to FIG. 2A, example operations for delivering electrical stimulation using an auditory prosthetic device are shown. At step 202, an acoustic stimulation is delivered to a subject. In some implementations, the acoustic stimulation is a singular or plurality of acoustic stimuli at different frequencies. In some implementations, the acoustic stimulation is a multitone stimulus (i.e., a signal combining multiple tones).

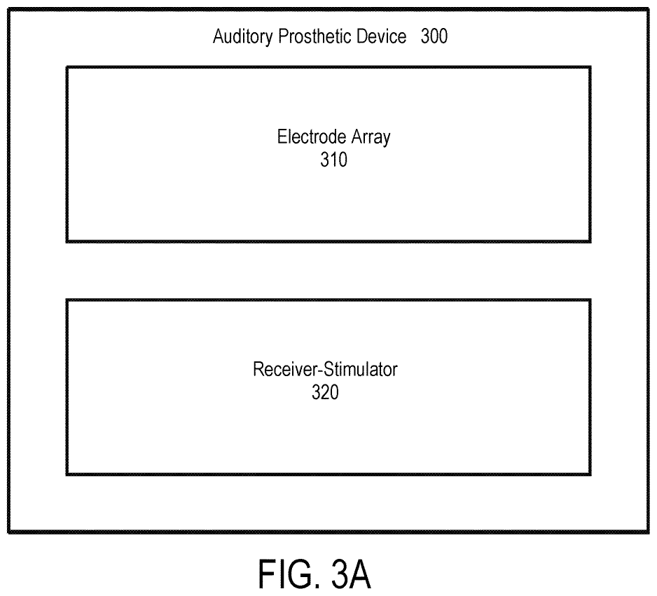

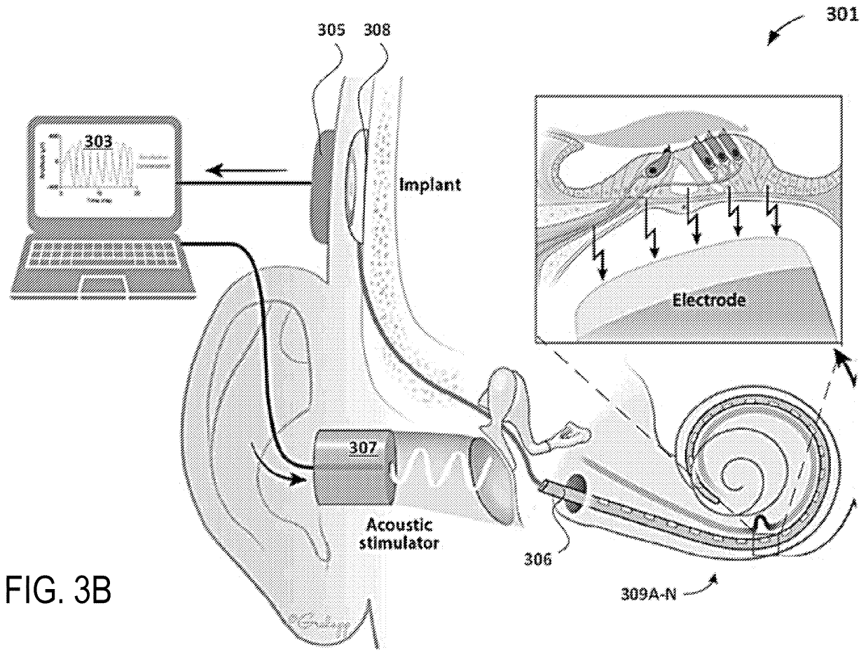

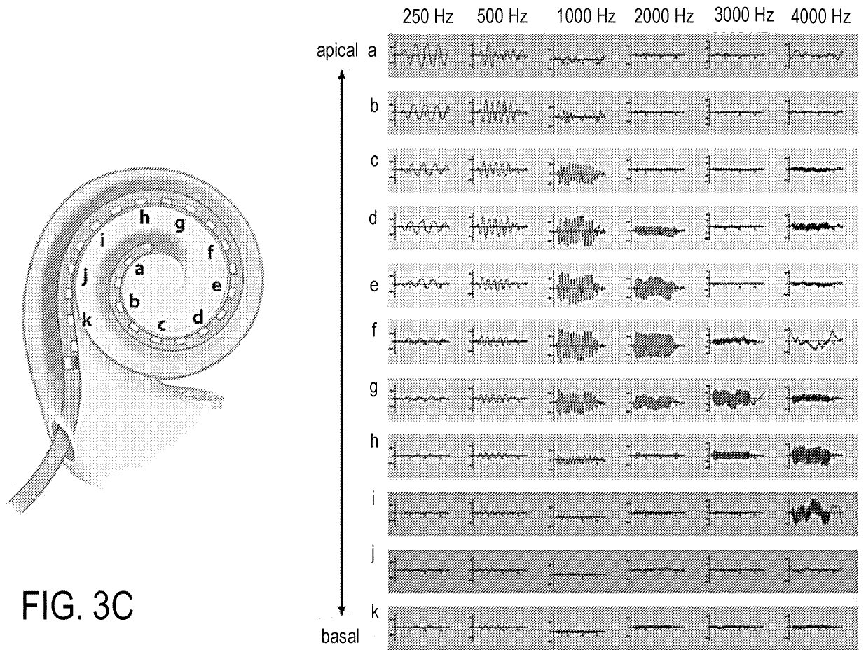

At step 204, an acoustically-evoked response to the acoustic stimulation is recorded using an electrode array. The acoustically-evoked response is recorded using the electrode array. This disclosure contemplates that the electrode array is implanted, at least partially, in the subject's ear. In the examples herein, the electrode array is multi-electrode array equipped with 22 electrode contacts. It should be understood that the number of electrode contacts is provided only as an example. Additionally, the angular position of each electrode contact can be measured using imaging (e.g., computed tomography (CT) imaging). Such an electrode array is surgically implanted into at least a portion of the subject's cochlea. It should be understood that the exact locations of the elements of the electrode array, relative to the tonotopic arrangement of the cochlea, are unknown after surgical insertion. Optionally, in some implementations, the electrode array is a component of an auditory prosthetic device such as an implantable or semi-implantable device. Optionally, such auditory prosthetic device is a cochlear implant. Optionally, in some implementations, the electrode array is a component of an auditory prosthetic device shown in FIG. 3A. It should be understood that FIG. 3A is only provided as an example. The acoustically-evoked response is an electrocochleography (ECochG) signal. For example, the acoustically-evoked response can be an early auditory potential including, but not limited to, a cochlear microphonic (CM), a compound action potential (CAP), a summating potential (SP), or an auditory nerve neurophonic (ANN).

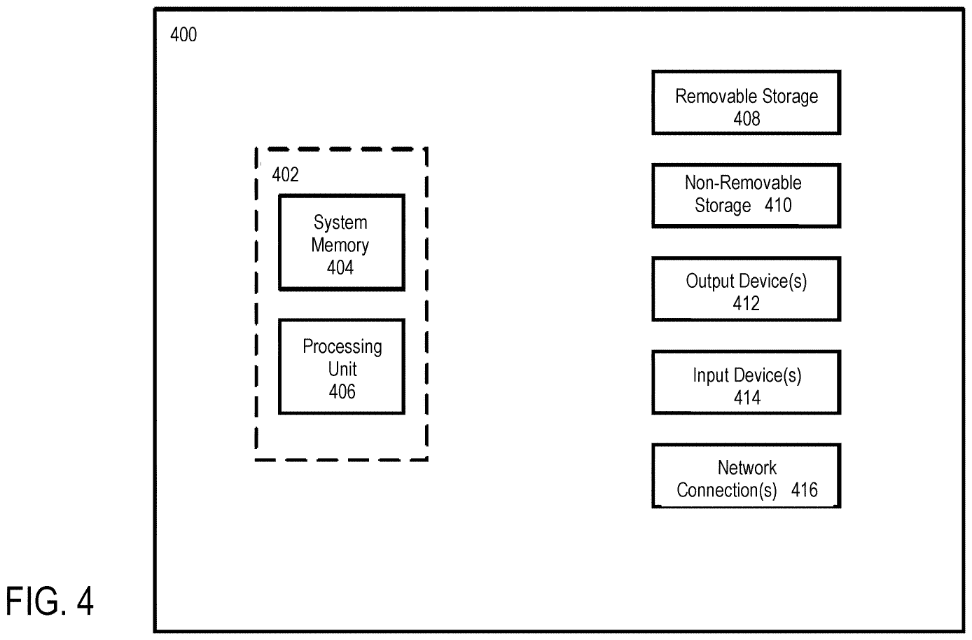

At step 206, the acoustically-evoked response is analyzed to determine a frequency response map, where the frequency response map associates respective frequencies of the acoustic stimulation to respective elements of the electrode array and underlying cochlear place or location. This includes localizing one or more characteristic frequencies (CF) or best frequencies (BF). A characteristic frequency (CF) or best frequency (BF) of a particular place along the basilar membrane is the frequency that peaks in response at that point. As described herein, CF is derived at threshold. For example, as described above with regard to FIGS. 1A-1C, ECochG recordings (e.g., which can be recorded at step 204) are used to directly measure and localize CF and BF. This disclosure contemplates analyzing at least one characteristic of the acoustically-evoked response including, but not limited to, time and/or frequency domain characteristics of the response such as amplitude and phase of the response. For example, the characteristics may include the amplitude and/or phase of the CM, ANN, SP, CAP, or combinations thereof. Alternatively, or additionally, this disclosure contemplates analyzing a physiological feature of the subject such as cochlear cell survival. The acoustically-evoked response is analyzed using a computing device such as the computing device shown in FIG. 4. Optionally, in some implementations, the computing device is part of the auditory prosthetic device, for example, as shown in FIG. 3A. In other implementations, the computing device is not part of an auditory prosthetic device, for example, a laptop, desktop, or tablet computer at a medical facility or clinic and/or another remote computer.

This disclosure contemplates that the response map can optionally be stored in memory of a computing device. Alternatively, or additionally, the response map can optionally be accessed by a computing device. In some implementations, the computing device is part of the auditory prosthetic device, for example, as shown in FIG. 3A. In other implementations, the computing device is not part of an auditory prosthetic device, for example, a laptop, desktop, or tablet computer at a medical facility or clinic and/or another remote computer. The response map can then be used when delivering electrical stimulation to the subject. For example, an acoustic signal can be recorded by a microphone of the auditory prosthetic device and then the acoustic signal can be converted to a digital signal. Thereafter, an electrical stimulation can be generated based on the digital signal. The electrical stimulation (e.g., one or more pulses) is characterized by at least one of a frequency, an amplitude, or a duration. As described below, such electrical stimulation can be delivered to the subject via the electrode array in a frequency-specific manner.

At step 208, an electrical stimulation is delivered to the subject via the respective elements of the electrode array based on the response map. The electrical stimulation is delivered using the electrode array of the auditory prosthetic device. In some implementations, the auditory prosthetic device is an implantable or semi-implantable device. Optionally, the auditory prosthetic device is a cochlear implant. As noted above, such an electrode array is surgically implanted into at least a portion of the subject's cochlea. Thus, the electrical stimulation is delivered in a frequency-specific manner to locations of the subject's cochlea via the respective elements of the electrode array. Accordingly, using the response map, the delivery of electrical stimulation accounts for the actual position of each element in the electrode array after surgical insertion, thus minimizing or eliminating partially or entirely the “frequency-to-place” mismatch that is a problem of conventional cochlear implants.

Example operations for localizing characteristic frequency (CF) or best frequency (BF) for cochlear implant mapping are also described herein. The method includes delivering acoustic stimulation to a subject, and recording, using an electrode array, an acoustically-evoked response to the acoustic stimulation, where the acoustically-evoked response is an electrocochleography (ECochG) signal. This disclosure contemplates that the electrode array is implanted, at least partially, in the subject's ear. In the examples herein, the electrode array is multi-electrode array equipped with 22 electrode contacts. It should be understood that the number of electrode contacts is provided only as an example. Additionally, the angular position of each electrode contact can be measured using imaging (e.g., computed tomography (CT) imaging). The method also includes analyzing the ECochG signal to determine a response map, where the response map associates respective frequencies of the acoustic stimulation to respective elements of the electrode array (and thus, underlying cochlear place). As described herein, the response map can be used to program an auditory prosthetic device such as an implantable or semi-implantable device. An example auditory prosthetic device is described with regard to FIG. 3A. Optionally, the auditory prosthetic device is a cochlear implant. As described herein, the response map determined by analyzing the ECochG signal can be used to control delivery of electrical stimulation in a manner that accounts for the actual position of each element in the electrode array after surgical insertion.

This disclosure contemplates analyzing the ECochG signal by performing a spectral analysis. For example, the Fast Fourier Transform (FFT) algorithm can be used to perform a spectral analysis of the ECochG signal. It should be understood that the FFT algorithm is only provided as an example. This disclosure contemplates using other algorithms for the spectral analysis including, but not limited to, Discrete Cosine Transform (DCT) or Short-Time Fourier Transform (STFT). In some implementations, the spectral analysis can be used to localize a plurality of characteristic frequencies (CF) or best frequencies (BF). In some implementations, the spectral analysis can be used to analyze at least one characteristic of the ECochG signal. In some implementations, the spectral analysis can be used to analyze a physiological feature of the subject.

Referring now to FIG. 2B, a flowchart depicting example operations for determining an auditory stimulation protocol for a subject is provided. The auditory stimulation protocol can be or comprise a response map, a table, a graph, or any other data entity describing relationships and/or associations between frequencies (e.g., frequency bands) of acoustic stimulation and particular electrodes (e.g., one or more electrodes in an electrode array).

At step 212, the method comprises delivering an acoustic stimulation to a subject, for example, via an electrode array. This disclosure contemplates that the electrode array is implanted, at least partially, in the subject's ear. In the examples herein, the electrode array is multi-electrode array equipped with 22 electrode contacts. It should be understood that the number of electrode contacts is provided only as an example.

Additionally, the angular position of each electrode contact can be measured using imaging (e.g., computed tomography (CT) imaging).

At step 214, the method comprises obtaining an acoustically-evoked response to the acoustic stimulation. For example, The acoustically-evoked response can be recorded using the electrode array. The acoustically-evoked response can be an (ECochG) signal and/or an early auditory potential such as a CM, CAP, SP or an ANN.

At step 216, the method comprises determining one or more stimulus parameters from the acoustically-evoked response. For example, various intensities, locations, and/or frequencies corresponding with particular stimulation (e.g., sounds, speech, or the like).

At step 218, the method comprises determining an auditory stimulation protocol for the subject based at least in part on the one or more stimulus parameters. In some implementations, the auditory stimulation protocol comprises an assignment of a respective frequency band to one or more electrodes. It should be understood that such assignments may including a given electrode being associated with more than one frequency or frequency band. By way of example, a first frequency band can be associated with electrodes ‘e1’ and ‘e2’ and a second frequency band can be associated with electrodes ‘e2,’ ‘e3,’ and ‘e4.’

The auditory stimulation protocol can be used to program an auditory prosthetic device for the subject. The auditory prosthetic device can be an implantable or semi-implantable device. An example auditory prosthetic device is described with regard to FIG. 3A. Optionally, the auditory prosthetic device is a cochlear implant.

### Example Auditory Prosthetic Device

Referring now to FIG. 3A, an example auditory prosthetic device 300 is described. The auditory prosthetic device 300 can include an electrode array 310 that is configured for implantation into a subject's inner ear, and a receiver-stimulator 320 operably coupled to the electrode array 310. For example, the electrode array 310 can be inserted into at least a portion of the subject's cochlea. This disclosure contemplates that the electrode array 310 can record early auditory potentials either inside or outside of the subject's cochlea. In some implementations, the electrode array 310 is partially inserted into the subject's cochlea. In other implementations, the electrode array 310 is completely inserted into the subject's cochlea. Additionally, in some implementations, the receiver-stimulator 320 is optionally implanted in the subject's body, while in other implementations, the receiver-stimulator 320 is located externally with respect to the subject's body. The electrode array 310 and the receiver-stimulator 320 can be coupled by a communication link. This disclosure contemplates the communication link is any suitable communication link. For example, a communication link may be implemented by any medium that facilitates signal exchange between the electrode array 310 and receiver-stimulator 320. In some implementations, the auditory prosthetic device 300 is a cochlear implant. It should be understood that the auditory prosthetic device 300 can be an implantable device such as a fully-implantable prosthetic device or a semi-implantable prosthetic device.

Additionally, the auditory prosthetic device 300 further includes a microphone (not shown), which is located externally (or internally) respect to the subject's body, that records sound, which is then processed by a sound/speech processing unit. In conventional devices, the sound/speech processing unit is worn by the subject (e.g., clipped to clothing or hooked behind the ear) and also located externally with respect to the subject's body, and the processed sound signal is then transmitted to the receiver-stimulator, which is implanted inside the subject's body. In these implementations, the microphone and/or sound/speech processing unit can be coupled to the implanted receiver-stimulator 320. The receiver-stimulator 320 then converts the processed sound signal into a stimulation signal, which is transmitted to the electrode array 310 arranged within the subject's cochlea. Thus, the electrode array 310 in a cochlear implant is driven by sound recorded by an external microphone. In some implementations, the auditory prosthetic device 300 can be embodied as a fully implanted device having an internally implanted microphone, speech processor, and/or power source (e.g., battery).

The electrode array 310 can include a plurality of electrodes (sometimes referred to herein as “contacts” or “elements”). The electrodes of the electrode array 310 can be arranged to correspond to different tonotopic locations within the subject's cochlea. It should be understood that the cochlea allows perception of sounds in a wide frequency range (e.g., ~20 Hz to ~20 kHz). Different portions of the cochlea move in response to different frequencies, for example, lower frequencies cause preferential movement and neural activation near the apex while higher frequencies cause preferential movement and neural activation near the base. Each of the electrodes of the electrode array 310 therefore records a different spectral component due to its respective tonotopic location. This disclosure contemplates that a respective potential can be recorded at each of the one or more electrodes. As described herein, the electrode array 310 can record the early auditory potential within the subject's cochlea, e.g., the electrical potential that arises naturally in the subject's cochlea through activity of sensory cells and auditory neurons. The early auditory potential can include cochlear microphonic (CM), which is produced by sensory hair cells in the cochlea. It should be understood that CM can be the dominant component of the early auditory potential. The early auditory potential, however, can include other components, for example, other potentials arising naturally in the subject's cochlea. These other potentials can include, but are not limited to, a compound action potential (CAP), a summating potential (SP), an auditory nerve neurophonic (ANN), a total response, and/or combinations thereof.

The receiver-stimulator 320 can include the device's circuitry, including a processor. Optionally, the processor is a digital signal processor (DSP). A DSP is a specialized microprocessor (e.g., including at least a processor and memory as described with regard to FIG. 4) for signal processing. Signal processing can include, but is not limited to, analog-to-digital conversion (ADC), filtering, compression, etc. of analog signals such as those recorded by the microphone. DSPs are known in the art and are therefore not described in further detail herein. The DSP of the receiver-stimulator 320 can be configured to receive acoustic signals recorded by the microphone, process such acoustic signals to generate a stimulation signal, and transmit the stimulation signal to the electrode array 310. As described herein, the stimulation signal(s) can be applied within the subject's cochlea using the electrode array 310.

### Example System

Referring now to FIG. 3B, an example system 301 that includes a computing device 303 and an auditory prosthetic device according to an illustrative embodiment is provided. The system 301 may be configured to deliver an acoustic stimulation, obtain acoustically-evoked responses to the acoustic stimulation (e.g., via the auditory prosthetic device), and analyze the acoustically-evoked responses to determine one or more stimulus parameters, generate a response map, and/or the like for a given subject.

The auditory prosthetic device can include implantable (i.e., internal) and non-implantable (i.e., external) components. The implantable components include a receiver/stimulator 308 and an electrode array 306, which includes a plurality of electrodes 309A-N. In FIG. 3B, the electrode array 306 includes 22 electrodes. It should be understood that the electrode array 306 can include more or less than 22 electrodes, which is only provided as an example. In some implementations, the receiver/stimulator 308 (e.g., receiver-stimulator) is optionally positioned in a subperiosteal pocket, and a electrode array 306 is optionally inserted into the round window of the subject's ear.

The non-implantable components include a headpiece (or transmitter) 305. The headpiece 305 is operably coupled with the receiver/stimulator 308. For example, the headpiece 305 can be configured to transmit sound signals to the receiver/stimulator 308. The auditory prosthetic device also includes a speech processor. The speech processor is configured to sense sound with a microphone, decodes sound into its frequency and intensity components, and deliver electrical stimulation to the electrode array 306. As described herein, such electrical stimulation can be delivered using a response map (e.g., FIG. 2A) and/or according to an auditory stimulation protocol (e.g., FIG. 2B). In some implementations, the speech processor is external to the subject (i.e., non-implantable), for example, separate from or integrated with the headpiece 305. Alternatively, in some implementations, the speech processor is internal (i.e., implantable).

Additionally, the auditory prosthetic device includes a sound tube, phone, or speaker 307, which is operably coupled to an acoustic generator. This disclosure contemplates that an acoustic generator may be a separate component, part of the computing device 303, or part of the speech processor.

The complex signal response measured from each electrode 309A-N consists of the electrical activity from outer and inner hair cells and the spiral ganglion (inset). The components of the ECochG response (cochlear microphonic, summating potential, compound action potential, auditory nerve neurophonic) are analyzed (amplitude and phase) at each electrode 309A-N of the electrode array 306 to decipher an appropriate map for stimulation. Subsequently, an auditory prosthetic device can be programmed for a subject based at least in part on the determined one or more stimulus parameters and/or response map.

Referring now to FIG. 3C, a schematic diagram depicting results from a study that was conducted using the system depicted in FIG. 3B. In particular, FIG. 3C shows data derived from recordings for each of a plurality of electrodes in response to electrical stimulus. Post-hoc analysis of the ongoing response was performed off-line to separate the hair cell and neural components of the response. A significant response was defined as one whose magnitude exceeded the noise floor by 3 standard deviations. As depicted in FIG. 3C, acoustic stimuli were presented at 250, 500, 1000, 2000, 3000, and 4000 Hz and in vivo recordings were made at all 22 electrode contacts. The responses at all even electrodes are shown on the right panel where the most-apical electrode (electrode a) shows a large response for the 250 Hz stimulus and the responses along more basal electrodes show larger responses for higher frequencies. The electrophysiological properties of the human cochlea are demonstrated with greater electrical responses to higher frequencies at the basal end and larger responses to lower frequencies at the apex. These recordings were performed in 50 subjects, showing similar findings.

### Example Computing Device

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random-access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that cause the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

### Discussion

Environmental auditory stimuli are complex and encompass a wide range of sound frequencies. The ability to accurately discriminate these frequencies is crucial for effective human communication. Our peripheral auditory organ, the cochlea, features a unique structural layout termed ‘tonotopy’ or place coding, which plays a vital role in frequency discrimination. The auditory hair cells located in the basal (proximal) region of the cochlea, near the round window, are preferentially activated by high-frequency sounds. Conversely, the apical (distal) region's hair cells exhibit greater sensitivity to lower frequencies. The basilar membrane, along with other soft tissues within the cochlea, acts as a spectral analyzer, spatially separating sound waves based on frequency, leading to distinct points of maximum basilar membrane displacement with resulting hair cell and neural activation. Georg von Békésy was the first to shed light on this spatial specificity by frequency within the cochlea (1-3).

Building on this understanding, when presented with a pure tone stimulus, the basilar membrane undergoes a displacement that peaks at a distinct location before decreasing in amplitude sharply. This displacement results in a unique frequency-to-place map on the basilar membrane, where each cochlear location is optimally responsive to a specific frequency—known as the “best frequency” (BF) or characteristic frequency (CF) when derived at threshold. The path to understanding this tonotopic organization was significantly broadened by the detection of electrical potentials in response to sound, which stem from both the outer hair cells (i.e., cochlear microphonic—CM) and the cochlear nerve's action potential. These discoveries, demonstrated in cat and guinea pig models, have paved the way for our current understanding of sound transduction (4-8). By integrating von Békésy's anatomical and physical descriptions with these electrophysiological insights, a fundamental framework has been established for theorizing human sound perception.

Despite these advancements, the electrophysiological characteristics related to frequency discrimination have been largely identified in animal models, severely limiting our understanding of the mechanisms of tonotopy in living humans. The reliability of previous in vivo animal and ex vivo models have been impeded by several significant and specific challenges. First, the process of surgical alterations and histological processing introduces unavoidable artifacts and spatial discrepancies (1-3, 9, 10). Secondly, the absence of cochlear amplification in ex vivo models, which requires the action of outer hair cells to enhance sensitivity and frequency selectivity, hampers their efficacy (11-13). Thirdly, the anatomical and physiological differences between in vivo animal models and humans, coupled with the difficulty of deeply probing the cochlear lumen in these models, constrain their applicability (14-16). Lastly, ex vivo studies using cadavers do not account for the dynamic biological changes (e.g., cochlear amplifier) that are known to influence passive cochlear mechanics (17). Therefore, in vivo electrophysiological measurements within the human cochlea are essential for (1) advancing our knowledge of cochlear tonotopy (2) progressing cochlear implant and hearing augmentation technologies, and (3) improving our understanding of the underlying mechanisms related to auditory disorders.

The primary aim of this research was to elucidate the cochlear tonotopic map in living humans. To address this question, a multi-electrode array is positioned along the longitudinal axis of the cochlear lumen during cochlear implant surgery. This approach has been previously used in cochlear implant patients for assessing hearing preservation (18, 19) and cochlear health and associated speech perception outcomes (20, 21). The experiment was initiated by delivering a pure tone through a sound delivery tube into the ear canal, and then capturing the resulting electrophysiologic responses along the multi-electrode array. To comprehend the impact of intensity on the tonotopic map, the intensity was modulated from threshold up to high levels. Subsequently, the insertion of the array within the cochlea was evaluated to determine if it induced a perceptual change that would indicate a tonotopic shift. Additionally, the creation of an artificial ‘third-window’, a procedure frequently employed in ex vivo cadaveric and animal experiments, was examined to determine whether it could cause a shift in the tonotopic map. Remarkably, the examination of the in vivo tonotopic map in humans revealed that the frequency-position map at conversational sound levels differs significantly from the currently accepted tonotopic map (22-24).

### Experimental Results

### In Vivo Electrophysiology-Based Frequency-Position Mapping

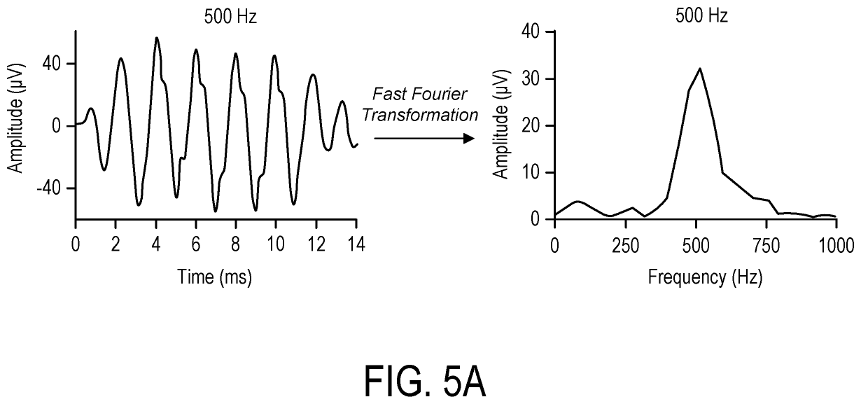

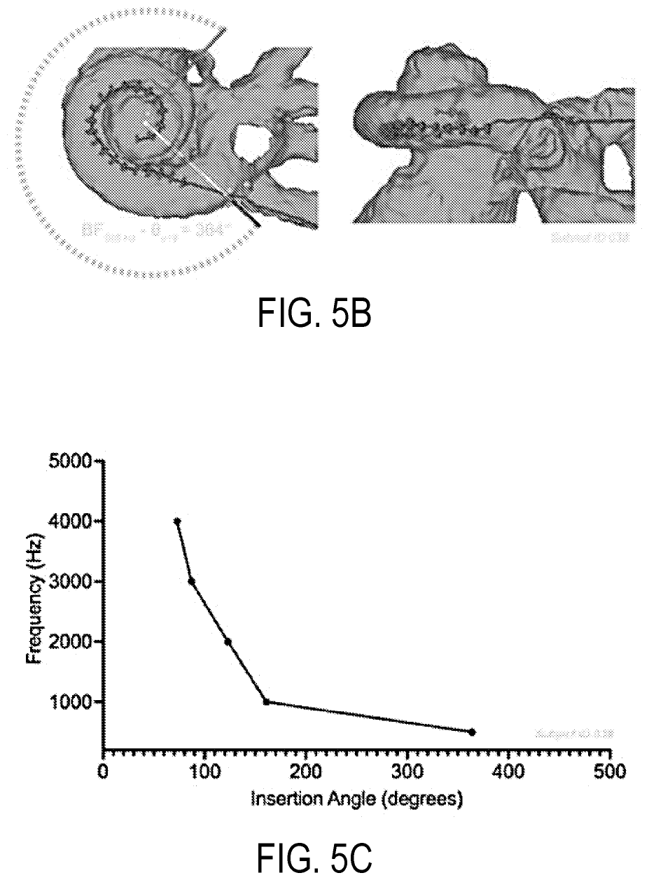

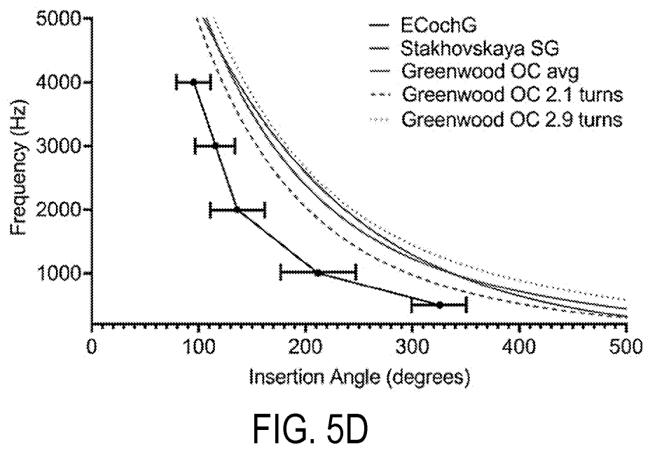

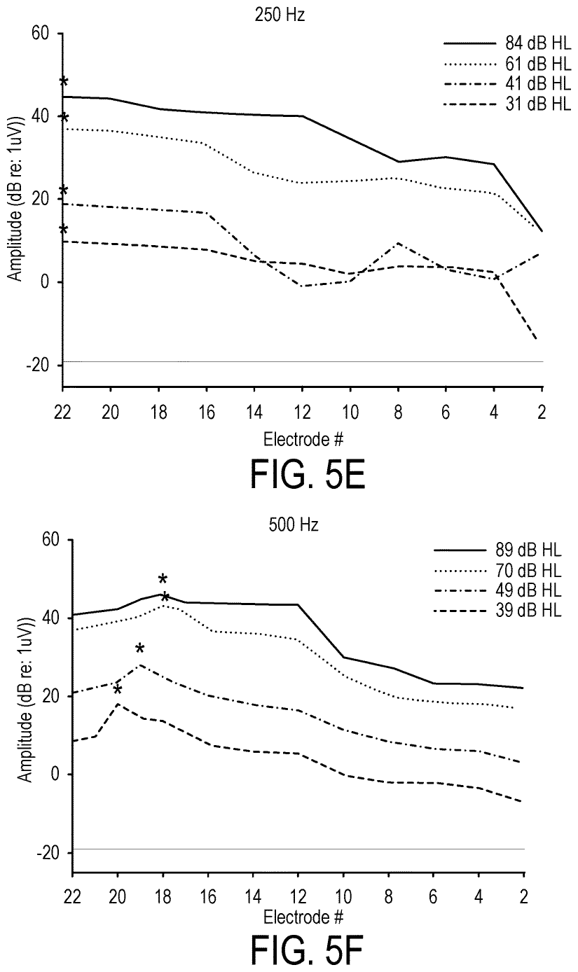

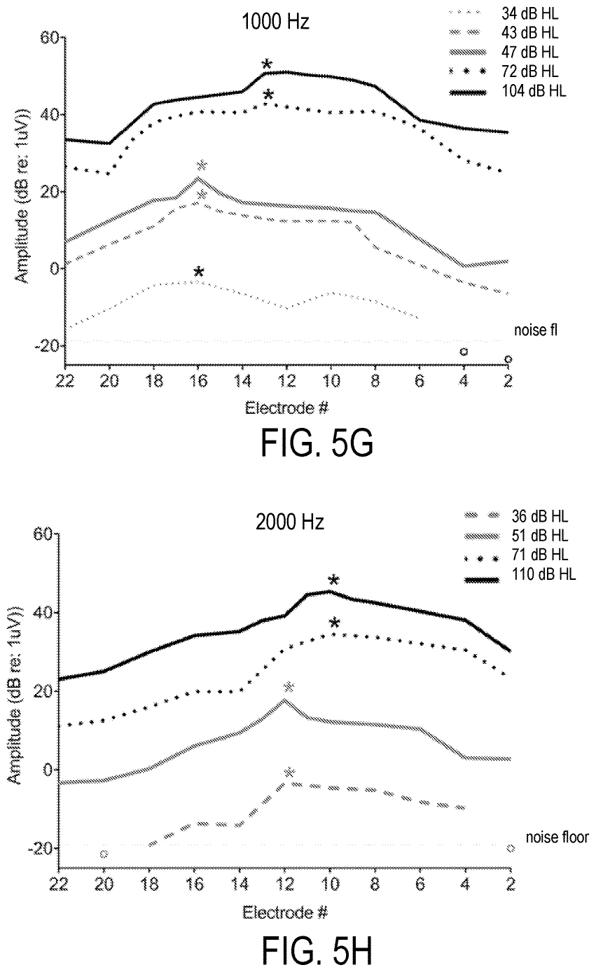

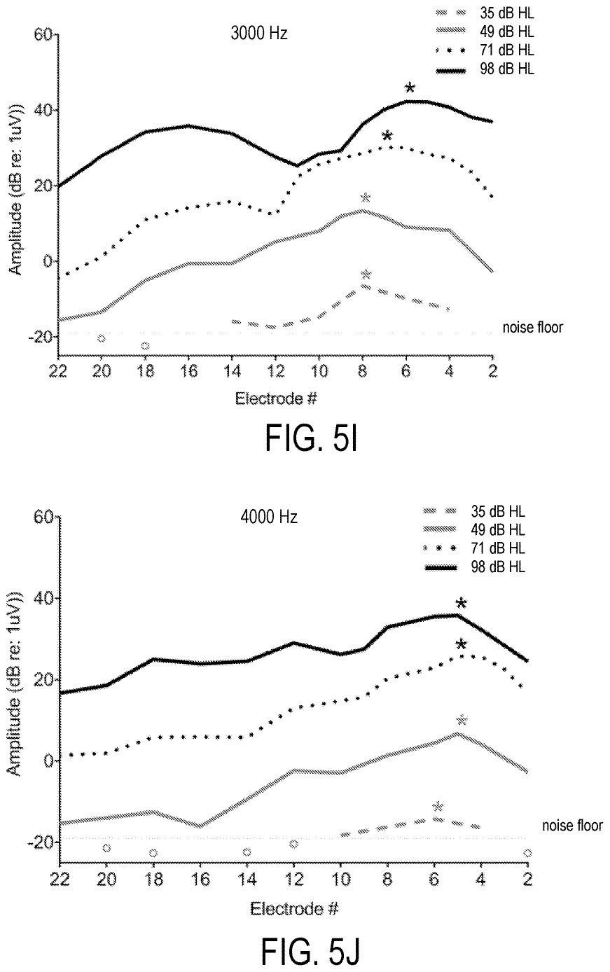

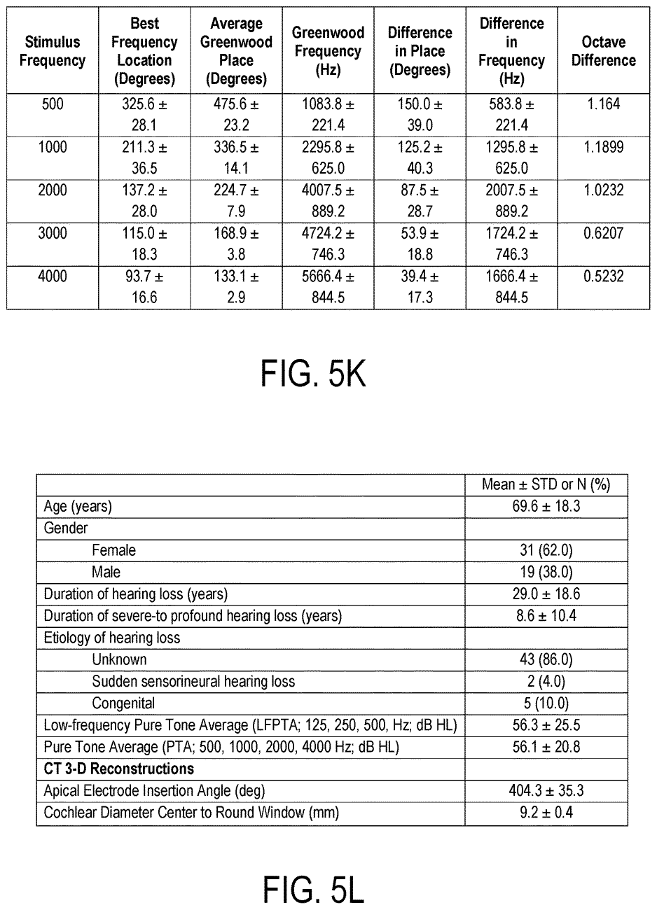

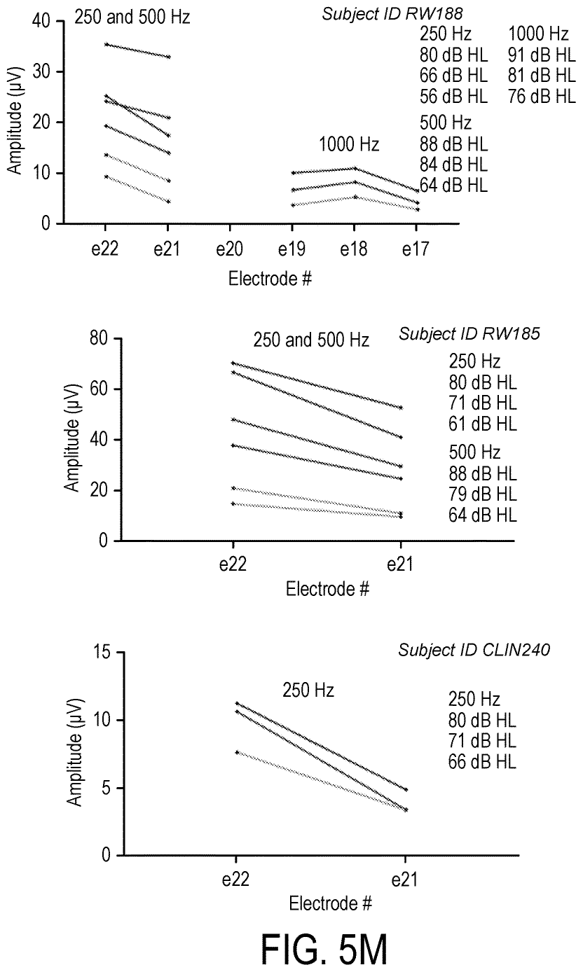

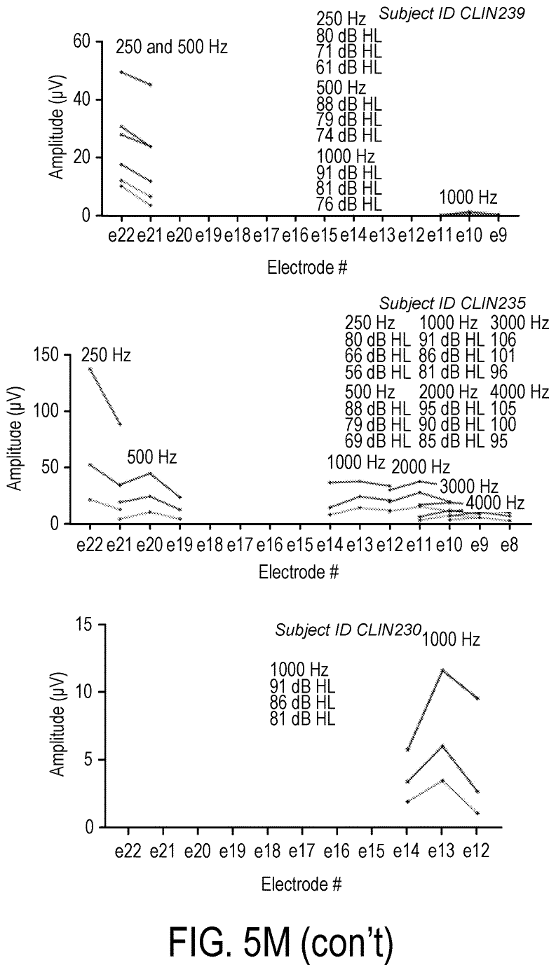

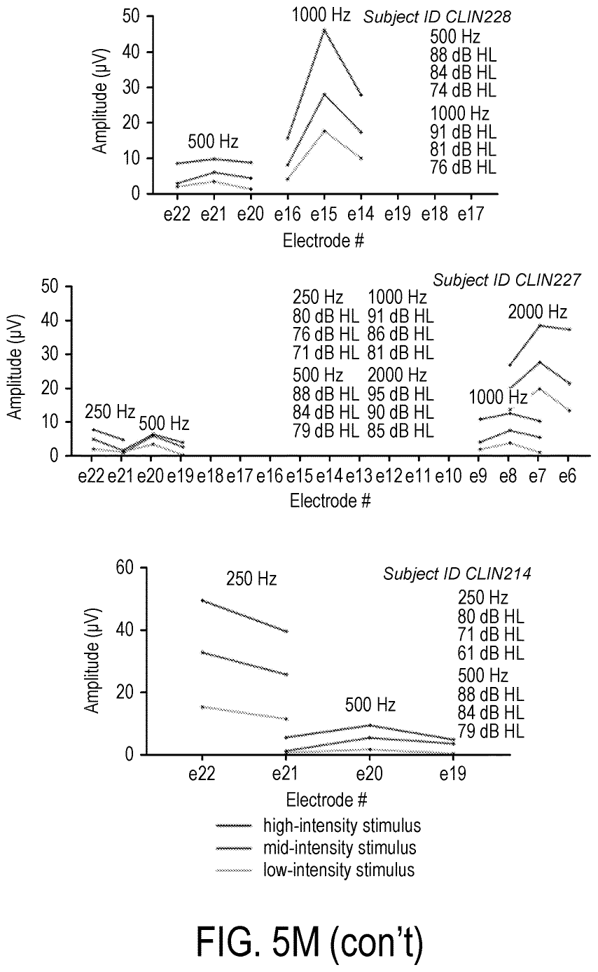

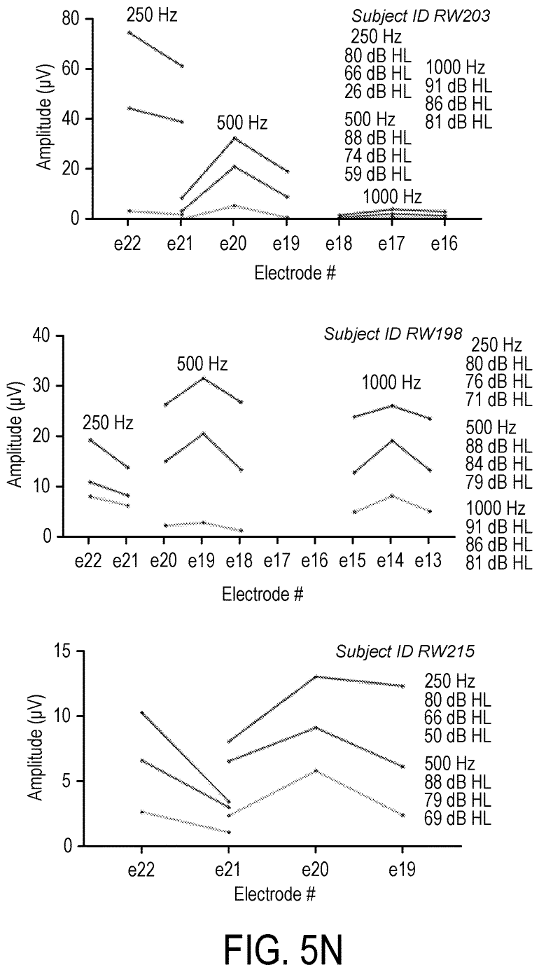

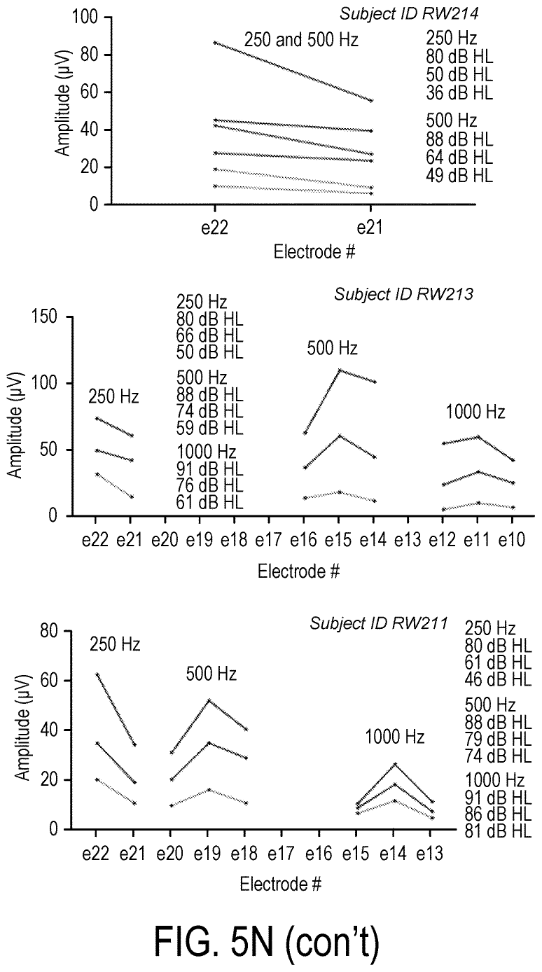

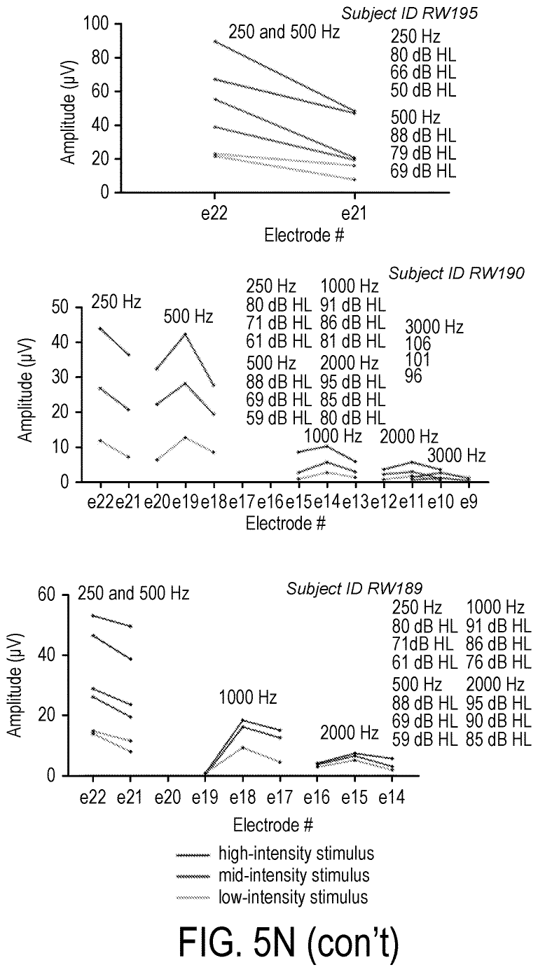

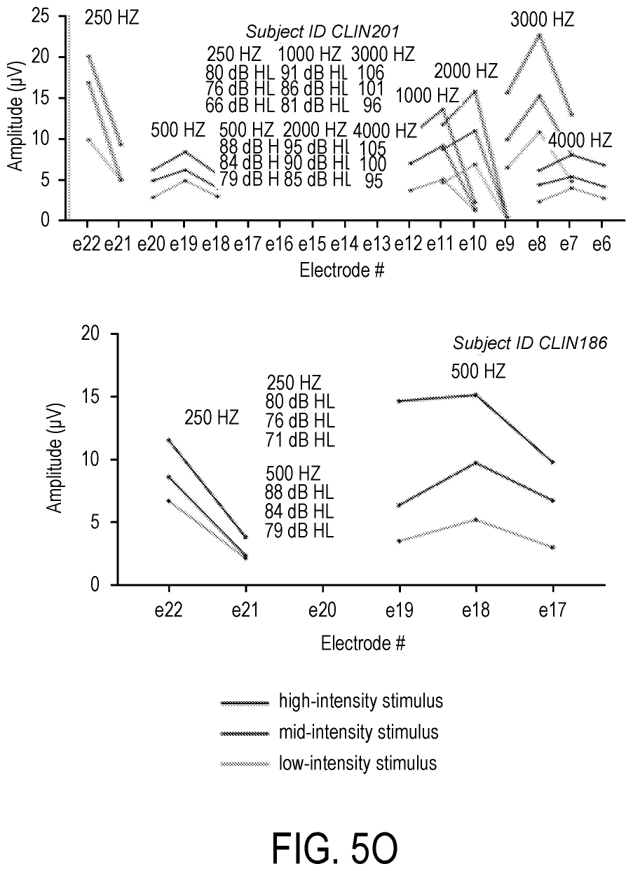

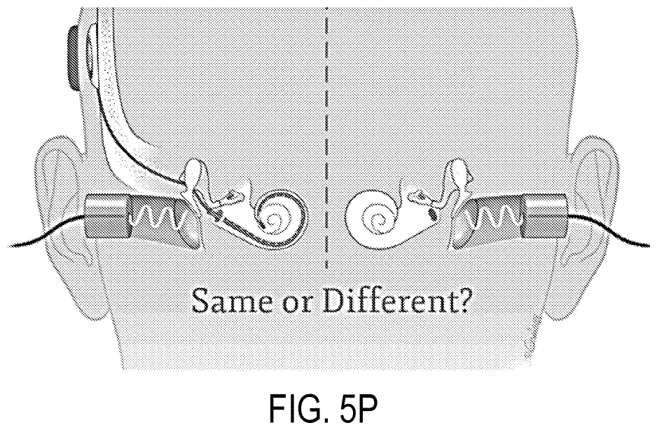

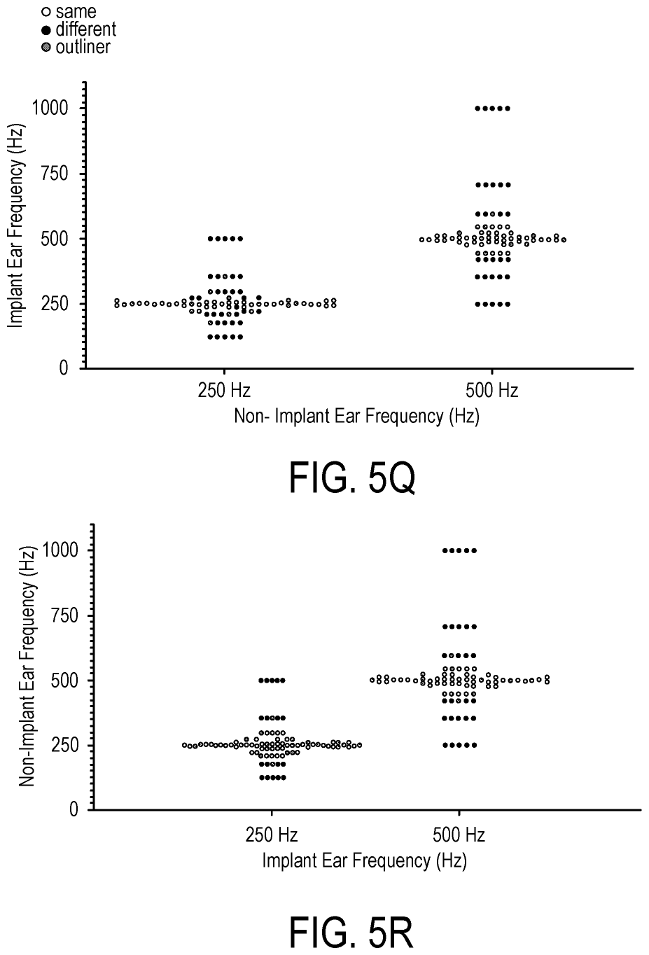

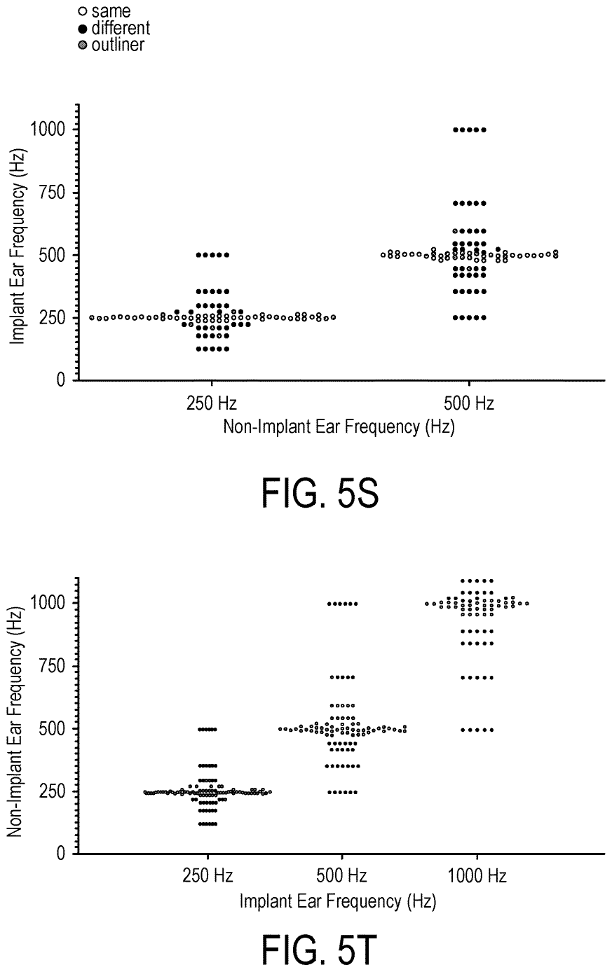

To construct an in vivo electrophysiological map, a multi-electrode array equipped with 22 platinum-iridium electrode contacts was implanted in 50 subjects. Following implantation, computed tomography (CT) imaging was utilized to accurately measure the angular position of each electrode contact, expressed in degrees. The mean number of cochlear turns across the cohort was 2.6, with a range from 2.2 to 2.9 turns. Demographics of all subjects are presented in FIG. 5L which shows demographic, audiologic, and imaging information of fifty subjects tested to construct aelectrophysiologically-derived frequency-position map. Acoustic tone-burst stimuli, ranging between 250 and 4000 Hz and alternating between rarefaction and condensation phases, were introduced immediately post-implantation. This was performed at suprathreshold intensities (~100 dB sound pressure level [SPL]). Evoked potentials were independently recorded across all 22 electrode contacts. The CM was primarily reflected by the calculated difference between the condensation and rarefaction phases as depicted in FIG. 3C. A fast Fourier transformation was applied to these difference waveforms at each electrode, and the amplitude of the first harmonic was evaluated (FIG. 5A). Using the first harmonic amplitude, CM tuning curves were generated across the electrode array for each subject. The electrode with the largest response on the CM tuning curve was designated the BF. The angular depth of the BF electrodes along the cochlear spiral, as determined by CT imaging, was plotted against the stimulus frequency (FIG. 5B, FIG. 5C, and FIG. 5D).

FIG. 5A is a graph showing a difference curve that was calculated by subtracting rarefaction from condensation phase stimuli. The difference consists consists primarily of the cochlear microphonic or the ongoing cyclical signal due primarily to the receptor current of outer hair cells. The ongoing portion of the response was selected for fast Fourier transformation and the amplitude of the response to the particular stimulus frequency was determined. Here, 500 Hz is shown for one subject. This was performed across all 22 electrodes in response to 500 Hz and the largest amplitude response was defined as the best frequency (BF) location for that particular frequency (e.g., BF 500 Hz). In this subject, the BF was at electrode-18 for 500 Hz.

FIG. 5B is a schematic diagram depicting Computed tomography (CT) imaging and 3D reconstructions that were performed postoperatively to identify the individual electrodes and visualize the adjacent soft tissue anatomy. To determine the position of each electrode, the CT image of each subject's cochlea was viewed along the mid-modiolar axis and the round window was marked at the 0° at the start of the cochlear canal since all insertions were performed at the round window. The angular position was then measured based on the rotation at the mid-modiolar axis. For this subject, the BF was at electrode-18 which was measured at 364° within the subject's cochlea. Here, the frequency-position relationship using electrophysiologic responses was determined for 500 Hz.

FIG. 5C is a graph showing results for the same methodology described above as performed for 1000, 2000, 3000 and 4000 Hz to develop a frequency-position function for the individual subject's cochlea using the electrophysiologic responses and CT imaging for the location of each electrode.

FIG. 5D is a graph showing results of electrophysiologic measurements repeated in 49 additional subjects. CT imaging was performed to identify the precise location of each BF to generate a cumulative frequency-position function for the electrophysiologically-derived map (ECochG map). Error bars are +/−2 standard deviations (SD). This was compared to organ of Corti (OC) and spiral ganglion (SG) maps as established by Greenwood and Stakhovskaya et al., respectively. The ECochG map in this study is at least one octave shifted downward in frequency or more basal in location compared to both the SG and OC maps. The OC map is also shown across various size cochleae (i.e., 2.1 and 2.9 turns), illustrating that the size variability cannot account for the difference between the ECochG map and the OC map.

Comparison with Preceding Frequency-Position Maps

To assess how the in vivo human frequency-position map from the present study deviates from previous models, it was compared with the widely accepted organ of Corti (OC; Greenwood) and spiral ganglion (SG; Stakhovskaya) maps, (23, 25), (FIG. 5D). These referenced maps, which form the foundation for understanding cochlear tonotopy, have been recently refined using synchrotron radiation phase-contrast imaging, a technique that allows for enhanced measurements of the cochlea's helicotrema and hook region (22, 26). Both OC and SG maps preserve frequency separation at levels approximating response thresholds, with minimal divergence in regions where peripheral axons follow a radial trajectory. However, a significant divergence emerges at angles exceeding 600 degrees, a compression point of peripheral axons not reached by the most distal electrode in the conducted study (22).

In examining the five stimulus frequencies (500, 1000, 2000, 3000, and 4000 Hz), a consistent disparity was noted between the BF locations obtained using the in vivo map described herein and those estimated by the Greenwood function. FIG. 5K is a table providing a comparative analysis of in vivo and Greenwood frequency-position functions. For instance, the Greenwood function predicted the cochlear location for the 500 Hz stimulus to be at 475.6±23.2 degrees, while the in vivo measurements revealed an average BF place of 325.6+28.1 degrees (difference 150.0±39.0 degrees). The Greenwood frequency at the 500 Hz BF location was 1083.8±221.4 Hz, resulting in a frequency difference of 583.8±221.4 Hz (1.1 octaves). This frequency and place disparity between in vivo recordings and Greenwood tapered basally, reaching 39.4:17.3 degrees and 1666.4±844.5 Hz (0.52 octaves) for the 4000 Hz stimulus.

### Effects of Stimulus Intensity on Frequency Tuning

Previous in vivo animal studies have established that increased stimulus sound pressure levels (SPLs) can result in less sharp frequency tuning that shifts the BF position toward a more basal location or where a given location represents a lower BF frequency (27, 28). To further investigate this relationship in humans, the shifts in BF responses across varying stimulus levels in our cohort were examined.

Twenty subjects with moderate to profound residual hearing post-implantation were exposed to varying stimulus intensities, ranging from 36 dB HL to 91 dB HL across a frequency range of 250 Hz to 2000 Hz. Recordings were conducted at the BF electrode and adjacent electrodes to determine if intensity changes would shift the BF location. It was discovered that as the stimulus level increased, the response peak heightened in amplitude but maintained the same location across all frequencies and patients tested. Thus, the frequency tuning and BF location remained stable despite reductions in stimulus intensity, though these responses were limited due to residual hearing, necessitating high stimulus levels. FIG. 5M, FIG. 5N, and FIG. 50 illustrate stimulus intensity and frequency-position maps generated from evaluation of the impact of stimulus intensity on the frequency tuning curve of the cochlea in 20 studies. Once the best frequency (BF) electrode was identified at the highest intensity stimulus (as defined by the limit of the speaker) for a particular frequency, the stimulus intensity was decreased and measurements were performed at the BF electrode and immediately adjacent electrodes to determine whether there would be an apical shift or basal shift of the BF with decreasing stimulus intensity. The frequency tuning and location of the BF did not shift with decreases in stimulus intensity, albeit responses were limited by the amount of residual hearing which necessitated high stimulus levels, in 20 subjects that were tested.

### Assessing the Impact of Stimulus Intensity on Frequency-Position Map

The influence of stimulus intensity on cochlear frequency tuning was examined through electrophysiological recordings in 22 subjects, which included twenty from the initial pool of 50 subjects and two with auditory neuropathy spectrum disorder. After identifying the BF electrode at the highest intensity stimulus (determined by the speaker's limit) for a specific frequency, the stimulus intensity was reduced in 5-dB increments. These measurements were conducted at the BF electrode and the adjacent electrodes to ascertain whether a decrease in stimulus intensity would lead to an apical or basal shift of the BF. For these measurements, a single sweep was performed and the noise floor was set at ~1.0 μV. All other parameters related to stimulus and recording, including signal processing, were identical to the procedure described earlier.

In order to model the effects of stimulus intensity changes on BF place in a cochlea with significantly preserved hair cell function, two patients with auditory neuropathy spectrum disorder were selected. These patients exhibited both present distortion-product otoacoustic emissions (DPOAEs) between 2 and 8 kHz and auditory brainstem response waveforms characteristic of a CM with absent neural waveforms. Post electrode array placement, measurements were taken across every electrode along the array at high-intensity (~90 dB HL, conversation-level intensity (~70 dB HL), and near threshold (~20-30 dB HL) for five stimulus frequencies (500-, 1000-, 2000-, 3000, and 4000-Hz). For these subjects, thirty sweeps were performed, and the CM tuning curves were generated for varying intensities and frequencies.

Two subjects, diagnosed with auditory neuropathy spectrum disorder with present distortion product otoacoustic emissions (DPOAEs), represented exceptional cases in which to perform a similar analysis. Auditory neuropathy spectrum disorder is associated with substantial preservation of cochlear hair cell function, thereby avoiding the limitations of our other patients. In both these subjects, increasing the SPL resulted in a broadening peak with the BF location shifting in a basal direction (FIG. 5E, FIG. 5F, FIG. 5G, FIG. 5H, FIG. 5I and FIG. 5J). The tonotopic tuning derived from the intracochlear electrocochleography of a patient with auditory neuropathy spectrum disorder (characterized by significant preservation of cochlear hair cell function) was examined under varied stimulus intensities. The panels here illustrate apical shifts in frequency-position mapping at reduced stimulus levels for 500 Hz, 1000 Hz, 2000 Hz, 3000 Hz, and 4000 Hz frequencies. The depicted amplitudes correspond to the fast Fourier transformation amplitudes of the difference response, largely indicative of the cochlear microphonic tuning curve (outer hair cell tuning curve). Asterisks (*) denote the best frequency (BF) electrode for each frequency at a given stimulus intensity. Notably, response patterns at conversation-like stimulus levels mirrored those observed at peak stimulation levels, suggesting that frequency-position maps during conversation are more consistent with high-intensity, electrophysiologically-derived maps than those predicted by the Greenwood function.

Referring now to FIG. 5E, FIG. 5F, FIG. 5G, FIG. 5H, FIG. 5I, and FIG. 5J, graphs showing results of the impact of intensity stimulus on the frequency-position map at 200 Hz, 500 Hz, 1000 Hz, 2000 Hz, 3000 Hz, and 4000 Hz, respectively, are provided.

A subject with auditory neuropathy spectrum disorder was tested, a condition known to have substantial preservation of cochlear hair cell function as evidenced by present distortion-product otoacoustic emissions (2-8 kHz) and cochlear microphonics on auditory brainstem response testing. This was carried out to determine whether stimulus intensity modulation could account for the basal shifted tonotopic tuning derived from the intracochlear electrocochleography (FIG. 5D). Reduction in stimulus level to the limits of the equipment revealed the expected shift in an apical direction for all frequencies tested: 250 Hz in FIGS. 5E, 500 Hz in FIGS. 5F, 1000 Hz in FIGS. 5G, 2000 Hz in FIGS. 5H, 3000 Hz in FIG. 5I, and 4000 Hz in FIG. 5J. The amplitude shown in each graph are the fast Fourier transformation amplitudes of the difference response, which is primarily representative of the cochlear microphonic tuning curve (i.e., outer hair cell tuning curve). The asterisk (*) represents the best frequency (BF) electrode for each frequency and particular stimulus intensity. The stimulus levels more similar to conversational speech showed responses similar to those seen at highest stimulation level rather than those at threshold, which emphasizes that the frequency-position maps during conversation are more similar to the high intensity electrophysiologically-derived map rather than that described by the Greenwood equation.

Conversely, as the stimulus level was reduced to the equipment's limits, the expected apical shift, towards Greenwood and Stakhovskaya et al.'s specifications was observed (23, 25). Importantly, when tested with stimulation levels closer to everyday conversational speech (~70 dB HL), responses aligned more closely with those observed during high-level stimulation (i.e., basal shifted) rather than those at threshold. Together, these data suggest the human cochlea's operating point during typical listening conditions is likely better represented by a map derived from high-intensity stimulation.

### Impact of Electrode Array on Frequency-Position Map

A study was conducted to explore the potential impact of the electrode array on the frequency-position map. Pure tone acoustic stimuli ranging from 125 to 1250 Hz were presented to both ears. To ensure equal loudness across frequencies, the acoustic stimuli between both ears was balanced using a seven-point loudness scale, ranging from inaudible to uncomfortably loud (29). One ear was held constant as the reference, delivering a single pure tone (either 250, 500, or 1000 Hz), while presenting the contralateral ear with pure tones in a random sequence. Subjects were asked to determine whether the pitches presented separately to each ear sounded the ‘same’ or ‘different’. FIG. 5P is a schematic diagram depicting pitch-discrimination testing to determine the impact of the presence of electrode on the frequency-position map. Pitch comparisons were obtained between acoustic stimuli presented sequentially to both the non-implanted ear and the implanted ear to determine whether the presence of the electrode had an impact on the acoustic-frequency position map. The cochlear implant processor was not used for this portion of the testing. Two subjects were selected for this portion of the testing who had residual acoustic hearing preserved in the implanted ear and similar hearing in the contralateral, non-implanted ear. The acoustic stimuli were first balanced for loudness at all of the tested frequencies. Then one ear was held constant as the reference where a single, brief pure tone was delivered alternating randomly with varying frequency pure tones in the contralateral ear. The subject was then asked to indicate whether the pitches presented to each ear sequentially sounded the ‘same’ or ‘different’.

Results showed a minor average difference (range, 1.30-1.65 semitones; 0.11-0.14 octaves) in the acoustic perception of pure tones between both ears in two subjects. Therefore, the perimodiolar electrode did not substantially affect the cochlea's acoustic frequency tuning. FIG. 5Q, FIG. 5R, FIG. 5S, and FIG. 5T are graphs showing results of the pitch-discrimination testing. In both subjects shown, FIG. 5Q, FIG. 5R, FIG. 5S, and FIG. 5T show the results where the non-implant ear was held constant and the acoustic stimulus was varied in the implant ear. The right graphs show the results where the implant ear was held constant and the non-implant ear was varied. The blue dots represent when the patient indicated that both pitches sound the same and the black dots represent when the patient indicated that both pitches sound different. The outliers in red were defined where the subject had one response that was different from the other 4 responses, which is commonly noted as a component of fatigue in pitch-discrimination testing. For subject 1 (FIG. 5Q and FIG. 5R), there was a mean 1.30 semitone difference for the acoustic perception of pure tones for both ears. For subject 2 (FIG. 5S and FIG. 5T), there was mean 1.65 semitone difference for both ears. This testing indicates that the perimodiolar electrode did not impact the acoustic frequency tuning of the cochlea to a degree that could explain the shift shown between the electrophysiologically-derived frequency-position and those previously-established for the organ of Corti and spiral ganglion (2D).

### Effects of Artificial ‘Third-Window’ on Frequency-Position Map

A study was conducted to investigate the potential influence of an artificial cochlear ‘third-window’ on the frequency position map using an exceptional case. This involved a subject with excellent residual hearing who was scheduled to undergo a translabyrinthine procedure for the resection of a vestibular schwannoma. Prior to labyrinthectomy, the electrode array was inserted into the cochlea's round window. Acoustically-evoked responses were then measured to determine the BF location across a range of 250 Hz to 4 kHz before and after a fenestration near the upper cochlear turns was created. The creation of the third-window did not result in any shift in the frequency-position map, suggesting that the recordings were not likely affected by any artifacts from the approach.

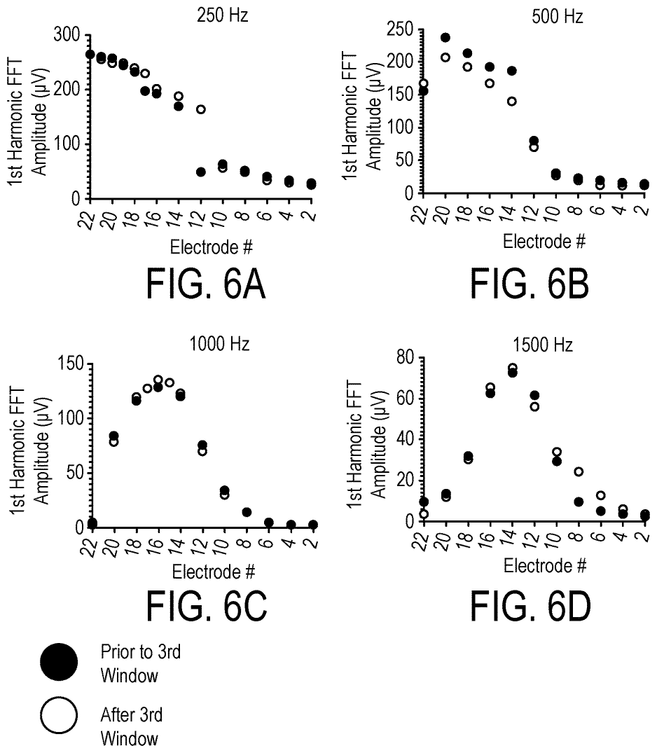

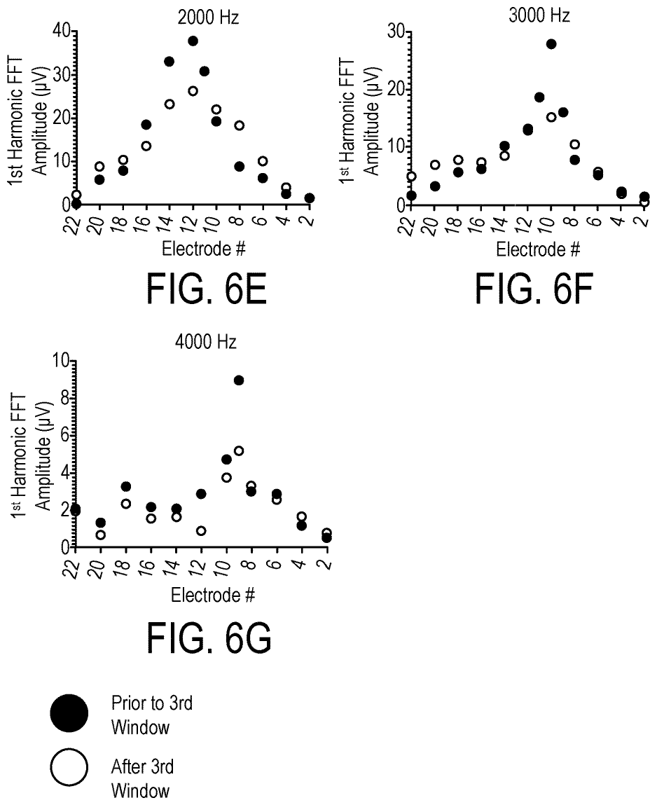

FIG. 6A, FIG. 6B, FIG. 6C, FIG. 6D, FIG. 6E, FIG. 6F, and FIG. 6G are graphs depicting the effects of third-window fenestration on the frequency-position map. Previous ex vivo experiments on tonotopy in humans have used a fenestration of the cochlear lumen to observe the traveling wave, but the potential impact of this artifact has not been studied. Impact of the third-window on electrophysiologic recordings was evaluated in a human subject with excellent residual hearing who was undergoing a translabyrinthine craniotomy (which intentionally destroys residual hearing) for resection of a vestibular schwannoma.

Prior to resection, the cochlea was approached identical to the electrophysiologic recordings performed previously and the best frequency (BF) was identified along the electrode array for 250-, 500-, 1000-, 2000-, 3000-, and 4000-Hz for the cochlear microphonic tuning curves (black dots in FIG. 6A, FIG. 6B, FIG. 6C, FIG. 6D, FIG. 6E, FIG. 6F, and FIG. 6G). Following the recordings, a 1-mm diamond burr was used to create a fenestration near the upper cochlear turns, and the CM tuning curves were generated for the individual frequencies (white dots in FIG. 6A, FIG. 6B, FIG. 6C, FIG. 6D, FIG. 6E, FIG. 6F, and FIG. 6G).

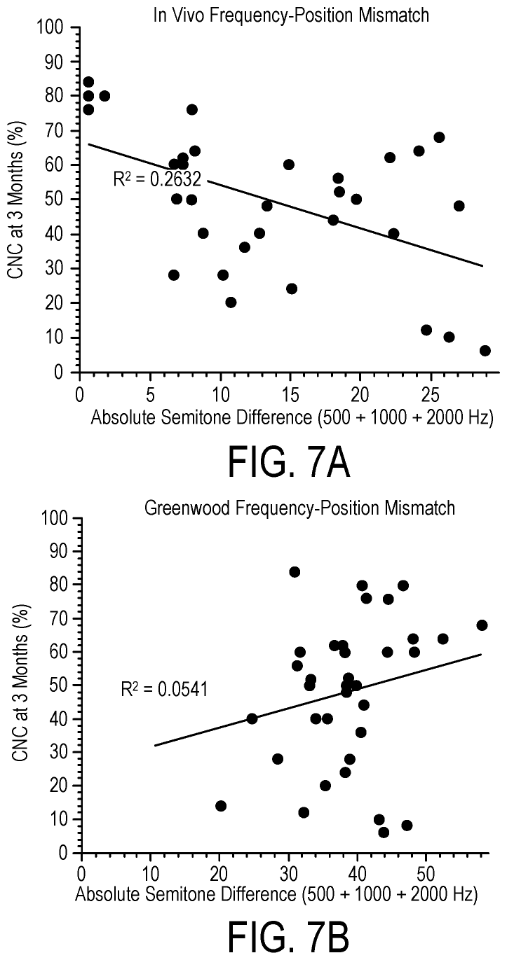

FIG. 7A and FIG. 7B are graphs depicting speech-perception performance results following cochlear implantation in relation to frequency-to-place mismatch between in vivo and Greenwood maps, respectively. The subjects of this study, for whom the in vivo map was developed using electrophysiologic recordings and imaging, were evaluated for their cochlear implant performance in a quiet environment at the three months post-activation.

The consonant-nucleus-consonant (CNC) word test was employed as an objective performance measure in quiet using the cochlear implant device. In FIG. 7A, the mismatch is correlated (in semitones) between each subject's default frequency allocation table at the best frequency electrode, as determined by electrophysiologic responses, with CNC word scores. A moderate linear correlation was observed, indicating that lower performance scores were associated with a greater mismatch. In FIG. 7B, the same best frequency electrode was compared with Greenwood's estimated frequency allocation within the same cohort. The calculated mismatch from Greenwood's calculation with the default frequency allocation table provided by the cochlear implant manufacturer is shown in FIG. 7B. No correlation between these variables was identified. These results reinforce the argument for employing in vivo electrophysiologic data for mapping and demonstrate that embodiments of the present disclosure may potentially enhance cochlear implant performance compared to the Greenwood map's place-based mapping approach.

### Discussion

### First-Ever Measurements of Human Electrophysiologically-Derived Frequency-Position Map

Embodiments of the present disclosure provide a novel approach to generating an accurate in vivo tonotopic map in humans with residual hearing, a task previously unattainable due to the delicate and inaccessible nature of the cochlea. The process of understanding cochlear tonotopy began with von Békésy (1, 2), who meticulously detailed the physical and anatomic observations of the cochlea in response to various tones in human cadavers. This was further advanced by Tasaki et al (4) and Wever et al (7, 8), who pioneered the measurement of electrical potentials from the cochlea in animals, discovering that these potentials were synchronized with the acoustic signal and were a consequence of hair cell stimulation.

Historically, direct measurements of mechanical or neural frequency tuning in cochleae were only feasible in laboratory animals, with assessments of the cochlea's basilar membrane vibrations largely limited to the basal high-frequency end where surgical access is more convenient (30). A study was conducted that leverages cochlear implantation as a unique model for analyzing cochlear mechanics in humans. The strategic placement of the multi-electrode array along the longitudinal axis of the cochlear lumen, in close proximity to residual hair cells and spiral ganglion neurons, enables the collection of robust acoustically-evoked responses. As the number of patients with significant residual acoustic hearing undergoing cochlear implantation increases, so does our ability to obtain substantial responses. This, in turn, allows for a more detailed characterization of the frequency channels established along the entire length of the cochlea, marking a significant advancement in the field of cochlear research.

In vivo Map Deviates from Standard Frequency-Position Functions at Conversational Intensity Levels

The in vivo map derived in the present study was subsequently compared to broadly-accepted frequency-position functions, specifically the Greenwood and Stakhovskaya maps (23, 25). Notably, an octave frequency downward discrepancy was observed (or basal direction shift) between the Greenwood map and the in vivo map in the frequency range of 500 to 2000 Hz, with smaller differences at higher frequencies (3 to 4 kHz). Several factors were explored to explain this shift, including stimulus intensity, presence of electrode, and creation of a third-window. It is important to note that all subjects in this study had underlying hearing loss, which necessitated their cochlear implants. Consequently, the electrophysiological findings presented here warrant recognition of this clinical context.

In mammalian species, outer hair cells play a crucial role as cochlear amplifiers, enhancing frequency selectivity and auditory sensitivity by up to 40 dB (31, 32). It is reasonably well-documented that high-level stimulation in animals can cause a half-octave shift of the tonotopic map in a basal or frequency-downward direction (33). Additionally, the subjects' existing otopathology in the present study could potentially impair the active cochlear mechanisms, leading to an additional shift in the tonotopic map (30). These factors likely contribute to our unexpected observation that reductions in stimulus intensity did not shift the tonotopic place coding of the cochlea as expected in most patients, except for the two patients with auditory neuropathy. In these patients, with better preserved amplifier functions, as evidenced by present DPOAEs, the anticipated effect of sound intensity modulation on place coding was observed.

Notably, when stimulus intensities were similar to everyday listening conditions (around 70 dB HL), the frequency-position responses aligned more closely with the high-intensity stimulus results, rather than those at threshold. This finding suggests that while our electrophythsiological results would likely align with the Stakhovskaya and Greenwood maps at threshold levels, the map derived from high intensity stimulation is likely more representative of the operating point of the human cochlea during everyday listening conditions.

### Electrode Array and Artificial Third-window Do Not Shift Frequency-Position Map

To ensure that the observed basal shift in the frequency-position map in our study was not a result of any intracochlear mechanical impact induced by the electrode array, interaural acoustic pitch comparisons were conducted. Although modeled by Kiefer et al (34), previous studies have yet to investigate the possibility of the electrode itself to induce a tonotopic shift in the frequency position function. If the electrode array itself was causing a shift in the map towards the basal end, a pitch in the implanted ear would be expected to be perceived at a lower frequency than in the ear without an implant. However, the conducted testing of human subjects did not uncover such a shift, suggesting the electrode was not artificially shifting the tonotopic map. However, it is worth noting that these findings may not apply to lateral wall electrodes, which have a higher likelihood of impacting the basilar membrane (compared to a perimodiolar electrode) due to their limited protection from the osseous spiral lamina (35).

Importantly, the creation of an artificial ‘third-window’ was investigated to determine whether it could potentially introduce an experimental artifact that altered the frequency-position map. While our in vivo recordings did not require a third-window, von Békésy, in his ex vivo observations of the traveling wave, created a fenestration along the bony labyrinth near the cochlear apex to visualize the waveform itself (1-3). Although von Békésy acknowledged the potential for some artifact, particularly at low frequencies, due to this apical fenestration, the impact of the third-window has not been thoroughly investigated in vivo within the human cochlea (1-3). In an experiment involving a single human subject, a third-window was created in a patient with good residual hearing who was undergoing resection of a large vestibular schwannoma via translabyrinthine craniotomy. Interestingly, there was no shift in the frequency-position map with the creation of the third-window, suggesting that the observed tonotopic discrepancy could not simply be explained by a third-window effect. These results also confirmed the robustness of the generated recordings and the underlying biological basis of the in vivo tonotopic map in humans.

### Implications for Implanted Auditory Prostheses

While the cochlear implant electrode array was utilized for recordings in this study, its primary function is an auditory prosthesis, designed to electrically stimulate the auditory nerve at a prescribed location. Prior research has underscored the importance of accurate tonotopic stimulation for speech comprehension in complex auditory environments (36, 37). The study findings are noteworthy since they closely align with those derived in single-sided hearing loss patients using cochlear implants where pitch perception in the normal ear was compared to those from electrically-stimulated contacts from known locations (38, 39).

Recently, discrepancies (or mismatch) between the default frequency allocation algorithms for the individual cochlear implant electrodes and Greenwood's function have been explored as an explanation for speech perception outcomes in patients (40). This research suggests that although the impact was quite small, greater degrees of mismatch (for frequency-to-place) might negatively influence speech perception outcomes. Mismatch between the generated in vivo map and the same default frequency allocation tables were compared and a moderate linear correlation was discovered where a larger mismatch resulted in poorer cochlear implant speech perception scores (FIG. 7A and FIG. 7B). When comparing the frequency-to-place mismatch against Greenwood's frequency-position function in our same patient cohort, no correlation was found with cochlear implant speech perception. These findings bolster the argument that the in vivo electrophysiologic map presented in the present disclosure is a far better representation of the actual operating tonotopic map than Greenwood's function. It is believed that this resulted from Greenwood's derivation at threshold responses rather than at conversational speech levels.

Embodiments of the present disclosure concern potential benefits of intensity-based mapping strategies, where different electrodes are activated based on the intensity of the acoustic stimulus, or a strategy that models the tonotopic map close to conversation levels to improve cochlear implant performance. Such strategies can enhance patient outcomes, providing a more effective and personalized approach to cochlear implantation.

In summary, the findings presented herein provide the first direct measure and derivation of an in vivo tonotopic map in humans. Notably, it was discovered that the map, at conversational levels, was shifted by nearly an octave compared to previously established frequency-position maps. This shift is a significant revision to our understanding of the tonotopic map compared with earlier studies. The immediate implication of our findings is for improved mapping and stimulation of cochlear implant electrodes, although there remain broad implications that extend beyond the advancement of cochlear implant and hearing augmentation technologies. These findings open new possibilities for exploring auditory disorders, speech processing, language development, and age-related hearing loss.

### Results

### Materials and Methods

Study Design and Objectives. A study was conducted that aimed to (1) construct a frequency-position function based on electrophysiological recordings and compare it with existing organ of Corti and spiral ganglion tonotopic maps of the human cochlea, and (ii) explore how variables such as stimulus intensity, presence of the recording electrode, and the creation of a third-window could influence the frequency-position map.

Participant Selection. Fifty participants were enlisted for this study, with the approval of the Institutional Review Board (IRB) of Washington University in St. Louis (IRB #202007087). Candidates for cochlear implantation were considered as potential participants. Eligibility criteria included adult individuals who possessed residual low-frequency hearing prior tosurgery, specifically a low-frequency puretone average of 125, 250, and 500 Hz ≤60 dB HL. Participants were excluded if they had middle ear pathology, were undergoing revision surgery, or if they lacked a patent external auditory canal, as the acoustic stimulus was delivered via air conduction. Candidates who were not English speaking or were unable to provide informed consent were also excluded.

Electrode Placement Surgical Procedure. Cochlear implant surgeries were conducted by a team of five experienced surgeons. A standard mastoidectomy-facial recess was used to gain access to the cochlea. Subsequently, the round window niche overhang was partially removed. Depending on the round window membrane orientation, the array was inserted either through a round window incision or after creation of a marginal cochlear opening. All insertions utilized a perimodiolar electrode array (Model CI632; Cochlear Corp., Sydney, NSW, Australia). An intraoperative radiograph confirmed expected coiling of the array. Post-insertion, the cochleostomy was sealed with temporalis muscle or fascia to avert perilymph leakage. The receiver-stimulator was securely positioned in a subperiosteal pocket.

Intracochlear Electrophysiological Measurements. Prior to the sterilization of the surgical site, an ER3-14A insert earphone (Etymotic, Elk Grove Village, IL, United States) was inserted into the external auditory canal. Once the electrode array was implanted into the cochlea, a telemetry coil was set over the skin, aligned with the cochlear implant antennae using a sterile ultrasound drape. All 22 electrodes within the array were conditioned in reference to the case ground to establish a common reference potential, minimize electrical noise interference, and ensure accurate and reliable measurements in the experimental setup. Tone burst stimuli at frequencies of 250, 500, 1000, 2000, 3000, and 4000 Hz were independently administered in both condensation and rarefaction phases, with a minimum of 30 repetitions per phase. The intensities for the respective frequencies were set at 108, 99.5, 98, 104, 102, and 101 dB HL, determined by the maximum output capacity of the speaker. Each stimulus had a duration of 14 ms with a rise and fall time of 1 ms, shaped by a Blackman window. The recording epoch was set to 18 ms, initiated 1 ms prior to stimulus onset, with a sampling rate of 20 kHz. The electrophysiological responses were recorded across all 22 electrodes of the array.

Electrophysiological Signal Analysis. The recorded electrophysiological responses, stored as separate condensation and rarefaction phases, were processed offline. Using custom software procedures in MATLAB R2020a (MathWorks Corp., Natick, MA, United States), the difference curve was calculated by subtracting the rarefaction phase stimuli from the condensation phase stimuli. From this difference curve, the ongoing portion of the response for fast Fourier transformation (FFT) was selected. This process facilitated determination of the amplitude of the response to the various stimulus frequencies. Utilizing these amplitudes, cochlear microphonic (CM) tuning curves were generated for each frequency across the entire electrode array. In line with previous studies, a significant response was defined as one where the magnitude exceeded the noise floor by three standard deviations (21, 41, 42). The noise floor for this series of recordings was approximately 0.3 μV.

### Determination of Electrode Position Using Computed Tomography Imaging

The position of all 22 electrodes along the implanted array was established through postoperative computed tomography (CT) scans and subsequent 3D reconstructions. The platinum iridium contacts cause a “bloom” effect on the CT image, complicating the identification of individual electrodes and adjacent soft tissue anatomy. To overcome this artifact, a validated technique for accurately pinpointing the position of the implanted electrodes within the cochlea (43-45) was employed. After co-registering each subject's pre-implant CT image with their post-implant CT image, electrode contacts were identified and segmented from the post-implant image data and copied onto the pre-implant image space. This composite image was used for subsequent analysis.

To visualize the scalar position of the electrode array and individual electrode contacts, the composite CT volume was aligned with a high-resolution micro-CT cochlear atlas, derived from cadaveric temporal bones (44). This reference was used to infer the location of soft tissue structures within the cochlea that are not resolved by conventional CT (e.g., basilar membrane). The composite CT volume of each subject's cochlea was viewed along the mid-modiolar axis to determine the position of each electrode. The round window was designated as the 0° starting point of the cochlear canal, since all electrode insertions were performed using a round window related approach. From this start point, the angular position of each electrode was measured based on rotation about the mid-modiolar axis.

### Electrophysiologically-Derived Frequency-Position Map

The “best frequency” (BF) refers to the specific electrode along the array-corresponding to a particular location on the basilar membrane-that yields the maximum response to a given frequency stimulus. The CT-derived angular mapping of the BF electrode was used for each stimulus to construct a tonotopic map in all 50 subjects. This electrophysiologically-derived map was then directly compared with the established organ of Corti and spiral ganglion frequency position map functions (22-24).

For a more detailed analysis, the electrophysiology-based map was directly compared to the Greenwood function, with each individual subject's specific cochlear size taken into account. The approach began with evaluating the angular location where the Greenwood function predicted the given frequency (250, 500, 1000, 2000, 3000, and 4000 Hz) to be located. Following this, the frequency according to Greenwood function was evaluated at the determined BF location by the electrophysiologically-derived map for the previously defined frequencies. Finally, the discrepancy between the actual and estimated location and frequency was computed, incorporating the octave difference into the calculation.

Additional experiments were conducted to examine the impact of stimulus intensity on the frequency-position map, to investigate pitch discrimination with the electrode array, and to assess the potential impact of a third-window on the frequency-position map. These analyses involved a subset of the subjects.

Examining Pitch Discrimination with Electrode Array

In order to investigate the electrode's influence on the frequency-position map, we conducted acoustic pitch-discrimination comparisons between ears in two subjects with unilateral cochlear implants. The cochlear implant processor was not utilized during this part of the test. Both subjects had been implanted for less than six months and had preserved residual hearing in the implanted ear after the operation (postoperative low-frequency pure tone average of 125-, 250-, and 500-Hz<60 dB HL). Furthermore, both subjects had comparable residual hearing in the non-implanted ear.

Each participant attended two sessions, with a minimum interval of two weeks between them. Each session lasted approximately two hours and took place in a double-walled sound booth. Audiometric air conduction thresholds ranging from 250 Hz to 8 kHz were recorded using insert earphones for each ear. First, the loudness of the acoustic stimuli was balanced at the tested frequency between ears, employing a seven-point loudness scale ranging from inaudible to uncomfortably loud (29). This provided confirm that the stimulus intensity was comfortable for both ears. Subsequently, the stimulus intensity of the non-implanted ear was kept constant at a comfortable level, while adjusting the acoustic stimulus intensity of the implanted ear in 5-dB and then 1-dB increments to pinpoint the exact stimulus level at which the tone sounded similar in both ears. This process was repeated three times for each frequency tested (250-, 500-, 1000-Hz), and the median value was used for the pitch-discrimination part of the test.

After achieving loudness balance at a comfortable level, the tested frequency (250-, 500-, 1000-Hz) was kept constant in the non-implant ear, while presenting the implant ear with acoustic pure tones that spanned 2 octaves from the non-implant ear's constant frequency, in a randomized order. A total of 80 individual presentations of various pure tones were made. The subject was then asked to specify whether the pitches presented to each ear separately sounded ‘same’ or ‘different’. Outliers were defined as instances where the subject's response deviated from the other four responses, an occurrence often attributed to fatigue in pitch-discrimination testing (45). This procedure was repeated in a separate session, but this time, the implant ear frequency remained constant while the non-implant ear frequency was varied.

### Assessing the Impact of a Third-Window on the Frequency-Position Map

Previous in vitro studies investigating tonotopy, including those conducted by von Békésy, have utilized a fenestration of the cochlear lumen for direct observation or recording of the traveling wave. However, the potential influence of this artifact on the frequency-position map remains unexplored. To assess the effect of a third-window on electrophysiologic recordings, a study was conducted involving a human subject with excellent residual hearing. This subject was undergoing a translabyrinthine craniotomy, a procedure that intentionally destroys residual hearing, for the removal of a vestibular schwannoma.

Before the labyrinthine resection, the cochlea was accessed and the same type of device as described above was inserted. The BF was identified along the array for stimulus frequencies of 250-, 500-, 1000-, 2000-, 3000-, and 4000-Hz at the maximum intensity output of the speaker. The signals were processed following the previously described methods. After generating the CM tuning curves, a 1-mm diamond burr was used to create a fenestration near the upper cochlear turns. Subsequently, CM tuning curves were generated again to investigate if the third-window had altered the location of the BF. Following these procedures, the implant was removed, and the surgery proceeded without complications. The CM tuning curves were generated for individual frequencies to allow a comparison between pre-and post-fenestration of a third-window.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The following patents, applications and publications, as listed below and throughout this document, are hereby incorporated by reference in their entirety herein.

### REFERENCES

1. G. J. N. L. Békésy, December, Concerning the pleasures of observing, and the mechanics of the inner ear. 11(1961 ).
2. G. Von Békésy, Zur Theorie des Hörens: Die Schwingungsform der Basilarmembran (Éditeur inconnu, 1928).
3. G. von Békésy, W. T. Peake (1990) Experiments in hearing. (Acoustical Society of America).
4. I. Tasaki, H. Davis, J. P. Legouix, The Space-Time Pattern of the Cochlear Microphonics (Guinea Pig), as Recorded by Differential Electrodes. The Journal of the Acoustical Society of America 24, 502-519 (1952).
5. H. Davis, The Electrical Phenomena of the Cochlea and the Auditory Nerve. The Journal of the Acoustical Society of America 6, 196-197 (1935).
6. H. Davis, A. J. Derbyshire, M. H. Lurie, L. J. Saul, THE ELECTRIC RESPONSE OF THE COCHLEA. American Journal of Physiology-Legacy Content 107, 311-332 (1934).
7. E. G. Wever, C. W. Bray, AUDITORY NERVE IMPULSES. Science (New York, N.Y.) 71, 215 (1930).
8. E. G. Wever, C. W. Bray, Action Currents in the Auditory Nerve in Response to Acoustical Stimulation. Proceedings of the National Academy of Sciences 16, 344-350(1930).
9. J. Pichat, J. E. Iglesias, T. Yousry, S. Ourselin, M. Modat, A Survey of Methods for 3D Histology Reconstruction. Medical Image Analysis 46, 73-105 (2018).
10. S. A. Taqi, S. A. Sami, L. B. Sami, S. A. Zaki, A review of artifacts in histopathology. Journal of oral and maxillofacial pathology: JOMFP 22, 279(2018 ).
11. D. T. Kemp, Stimulated acoustic emissions from within the human auditory system. The Journal of the Acoustical Society of America 64, 1386-1391 (1978).
12. W. S. Rhode, Some observations on cochlear mechanics. The Journal of the Acoustical Society of America 64, 158-176 (1978).
13. W. E. Brownell, C. R. Bader, D. Bertrand, Y. de Ribaupierre, Evoked mechanical responses of isolated cochlear outer hair cells. Science (New York, N.Y.) 227, 194-196 (1985).
14. P. M. Sellick, G. K. Yates, R. Patuzzi, The influence of Mossbauer source size and position on phase and amplitude measurements of the guinea pig basilar membrane. Hearing Research 10, 101-108 (1983).
15. N. P. Cooper, W. S. Rhode, Basilar membrane mechanics in the hook region of cat and guinea-pig cochleae: Sharp tuning and nonlinearity in the absence of baseline position shifts. Hearing Research 63, 163-190 (1992).
16. J. B. Nadol, Jr., Comparative anatomy of the cochlea and auditory nerve in mammals. Hear Res 34, 253-266 (1988).
17. Y. Zhang et al., Prestin derived OHC surface area reduction underlies age-related rescaling of frequency place coding. Hearing research 423, 108406 (2022).
18. A. Walia et al., Early Hearing Preservation Outcomes Following Cochlear Implantation With New Slim Lateral Wall Electrode Using Electrocochleography. Otol Neurotol 43, 443-451 (2022).
19. A. Walia et al., Is Characteristic Frequency Limiting Real-Time Electrocochleography During Cochlear Implantation? Frontiers in neuroscience 16, 915302 (2022).
20. A. Walia et al., Electrocochleography and cognition are important predictors of speech perception outcomes in noise for cochlear implant recipients. Scientific Reports 12, 3083 (2022).
21. A. Walia et al., Promontory Electrocochleography Recordings to Predict Speech-Perception Performance in Cochlear Implant Recipients. Otol Neurotol 43, 915-923 (2022).
22. L. Helpard et al., An Approach for Individualized Cochlear Frequency Mapping Determined From 3D Synchrotron Radiation Phase-Contrast Imaging, IEEE Trans Biomed Eng 68, 3602-3611 (2021).
23. D. D. Greenwood, A cochlear frequency-position function for several species—29 years later. J Acoust Soc Am 87, 2592-2605 (1990).
24. O. Stakhovskaya, D. Sridhar, B. H. Bonham, P. A. Leake, Frequency map for the human cochlear spiral ganglion: implications for cochlear implants. Journal of the Association for Research in Otolaryngology: JARO 8, 220-233 (2007).
25. O. Stakhovskaya, D. Sridhar, B. H. Bonham, P. A. Leake, Frequency Map for the Human Cochlear Spiral Ganglion: Implications for Cochlear Implants. Journal for the Association for Research in Otolaryngology 8, 220 (2007).
26. H. Li et al., Three-dimensional tonotopic mapping of the human cochlea based on synchrotron radiation phase-contrast imaging. Scientific Reports 11, 4437 (2021).
27. M. Chatterjee, J. J. Zwislocki, Cochlear mechanisms of frequency and intensity coding. I. The place code for pitch. Hear Res 111, 65-75 (1997).
28. I. J. Russell, K. E. Nilsen, The location of the cochlear amplifier: spatial representation of a single tone on the guinea pig basilar membrane. Proc Natl Acad Sci U S A 94, 2660-2664 (1997).
29. P. J. Blamey, L. F. Martin, Loudness and satisfaction ratings for hearing aid users. J Am Acad Audiol 20, 272-282 (2009).
30. L. Robles, M. A. Ruggero, Mechanics of the mammalian cochlea. Physiol Rev 81, 1305-1352 (2001).
31. M. C. Liberman et al., Prestin is required for electromotility of the outer hair cell and for the cochlear amplifier. Nature 419, 300-304 (2002).
32. R. Fettiplace, C. M. Hackney, The sensory and motor roles of auditory hair cells. Nature reviews. Neuroscience 7, 19-29 (2006).
33. M. A. Ruggero, N. C. Rich, A. Recio, The effect of intense acoustic stimulation on basilarmembrane vibrations. Auditory Neuroscience 2, 329-345 (1996).
34. J. Kiefer, F. Böhnke, O. Adunka, W. Arnold, Representation of acoustic signals in the human cochlea in presence of a cochlear implant electrode. Hear Res 221, 36-43 (2006).
35. F. Risi, Considerations and Rationale for Cochlear Implant Electrode Design—Past, Present and Future. The journal of international advanced otology 14, 382-391 (2018).
36. A. J. Oxenham, J. G. Bernstein, H. Penagos, Correct tonotopic representation is necessary for complex pitch perception. Proc Natl Acad Sci USA 101, 1421-1425 (2004).
37. M. K. Qin, A. J. Oxenham, Effects of simulated cochlear-implant processing on speech reception in fluctuating maskers. J Acoust Soc Am 114, 446-454 (2003).
38. J. P. M. Peters, E. Bennink, G. A. van Zanten, Comparison of Place-versus-Pitch Mismatch between a Perimodiolar and Lateral Wall Cochlear Implant Electrode Array in Patients with Single-Sided Deafness and a Cochlear Implant. Audiology and Neurotology 24, 38-48 (2019).
39. T. F. Tóth et al., Matching the pitch perception of the cochlear implanted ear with the contralateral ear in patients with single-sided deafness: a novel approach. Eur Arch Otorhinolaryngol 10.1007/s00405-023-08002-z (2023).
40. M. W. Canfarotta et al., Frequency-to-Place Mismatch: Characterizing Variability and the Influence on Speech Perception Outcomes in Cochlear Implant Recipients. Ear Hear 41, 1349-1361 (2020).
41. D. C. Fitzpatrick et al., Round window electrocochleography just before cochlear implantation: relationship to word recognition outcomes in adults. Otology & neurotology: official publication of the American Otological Society, American Neurotology Society [and] European Academy of Otology and Neurotology 35, 64-71 (2014).
42. N. H. Calloway et al., Intracochlear electrocochleography during cochlear implantation. Otol Neurotol 35, 1451-1457 (2014).
43. L. K. Holden et al., Factors affecting open-set word recognition in adults with cochlear implants. Ear and hearing 34, 342-360 (2013).
44. J. Teymouri, T. E. Hullar, T. A. Holden, R. A. Chole, Verification of computed tomographic estimates of cochlear implant array position: a micro-CT and histologic analysis. Otol Neurotol 32, 980-986 (2011).
45. M. W. Skinner et al., In vivo estimates of the position of advanced bionics electrode arrays in the human cochlea. The Annals of otology, rhinology & laryngology. Supplement 197, 2-24 (2007).
46. Walia et al. Place Coding in the Human Cochlea. medRxiv 2023.04.13.23288518; doi: https://doi.org/10.1101/2023.04.13.23288518

1. A method comprising:
delivering an acoustic stimulation to a subject;
recording, using an electrode array inserted into at least a portion of the subject's cochlea, an acoustically-evoked response to the acoustic stimulation;
analyzing the acoustically-evoked response to determine a response map, wherein the response map associates respective frequencies of the acoustic stimulation to respective elements of the electrode array; and
delivering, using the electrode array, an electrical stimulation to the subject, wherein the electrical stimulation is delivered via the respective elements of the electrode array based on the response map.

2. The method of claim 1, further comprising:
receiving an acoustic signal recorded by a microphone;
converting the acoustic signal to a digital signal; and
generating the electrical stimulation based on the digital signal.

3. The method of claim 1, wherein the electrical stimulation is delivered in a frequency-specific manner to locations of the subject's cochlea via the respective elements of the electrode array.

4. The method of claim 1, wherein the step of analyzing the acoustically-evoked response to determine the response map comprises localizing a plurality of characteristic or best frequencies.

5. The method of claim 1, wherein the step of analyzing the acoustically-evoked response to determine the response map comprises analy zing at least one characteristic of the acoustically-evoked response.

6. The method of claim 1, wherein the step of analyzing the acoustically-evoked response to determine the response map comprises analyzing a physiological feature of the subject.

7. The method of claim 1, wherein the acoustically-evoked response is an electrocochleography (ECochG) signal.

8. The method of claim 1, wherein the acoustically-evoked response is an early auditory potential.

9. The method of claim 1, wherein the electrical stimulation is characterized by at least one of a frequency, an amplitude, or a duration.

10. (canceled)

11. (canceled)

12. (canceled)

13. (canceled)

14. An auditory prosthetic device comprising:
an electrode array that is configured for insertion into at least a portion of a subject's cochlea; and
a processor coupled to the electrode array, wherein the processor is configured to:
store an acoustically-evoked response map, wherein the acoustically-evoked response map associates respective frequencies of an acoustic stimulation to respective elements of the electrode array;
receive an acoustic signal recorded by a microphone;
convert the acoustic signal to a digital signal;
generate an electrical stimulation based on the digital signal; and
transmit the electrical stimulation to the electrode array, wherein the electrical stimulation is transmitted to the respective elements of the electrode array based on the acoustically-evoked response map.

15. The device of claim 14, wherein the electrical stimulation is delivered in a frequency-specific manner to locations of the subject's cochlea via the respective elements of the electrode array.

16. The device of claim 14, wherein the electrical stimulation is characterized by at least one of a frequency, an amplitude, or a duration.

17. The device of claim 14, wherein the auditory prosthetic device is an implantable or semi-implantable device.

18. The device of claim 14, wherein the auditory prosthetic device is a cochlear implant

19. A method comprising:
delivering an acoustic stimulation to a subject;
obtaining an acoustically-evoked response to the acoustic stimulation;
determining one or more stimulus parameters from the acoustically-evoked response; and
determining an auditory stimulation protocol for the subject based at least in part on the one or more stimulus parameters.

20. The method of claim 19, wherein the auditory stimulation protocol is used to program an auditory prosthetic device for the subject.

21. The method of claim 19, wherein the auditory prosthetic device comprises a plurality of electrodes, and wherein the auditory stimulation protocol defines an association of a respective frequency to one or more electrodes.

22. The method of claim 19, wherein the stimulus parameters comprise at least one of an intensity, a frequency, and a cochlea location.
